(19) United States
(12) Patent Application Publication
(10) Pub. No.: US 2023/0390220 A1
(43) Pub. Date: Dec. 7, 2023

(54) MONOAMINE OXIDASE BLOCKADE THERAPY FOR TREATING CANCER THROUGH REGULATING ANTITUMOR T CELL IMMUNITY

(71) Applicant: THE REGENTS OF THE UNIVERSITY OF CALIFORNIA, OAKLAND, CA (US)

(72) Inventors: Lili Yang, Los Angeles, CA (US); Xi Wang, Los Angeles, CA (US)

(73) Assignee: THE REGENTS OF THE UNIVERSITY OF CALIFORNIA, OAKLAND, CA (US)

(21) Appl. No.: 18/249,663

(22) PCT Filed: Oct. 22, 2021
(86) PCT No.: PCT/US21/56181

§ 371 Date: Apr. 19, 2023

Related U.S. Application Data
(60) Provisional application No. 63/104,154, filed on Oct. 22, 2020.

(51) Int. Cl. A61K 31/15; A61K 31/5375; A61K 31/138; A61K 39/395; A61P 35/00

(52) U.S. Cl. CPC A61K 31/15; A61K 31/5375; A61K 31/138; A61K 39/3955; A61P 35/00

(57) ABSTRACT

Monoamine oxidase A (MAO-A) is an enzyme best known for its function in the brain, where it breaks down neurotransmitters and thereby influences mood and behavior. While small molecule MAO inhibitors (MAOIs) have been developed used for treating depression and other neurological disorder, the involvement of MAO-A in antitumor immunity has not been known. The disclosure provided herein identifies MAO-A as an immune checkpoint and the use of MAOI antidepressants for cancer immunotherapy. Here we report induction of the Maoa gene in tumor-infiltrating immune cells. MAOI treatment significantly suppressed tumor growth in preclinical mouse syngeneic and human xenograft tumor models in a T cell-dependent manner. Combining MAOI and anti-PD-1 treatments generated synergistic tumor suppression effects. Clinical data correlation studies associated intratumoral MAOA expression with T cell dysfunction and impaired patient survival in a broad range of cancers.

### CROSS-REFERENCE TO RELATED APPLICATIONS

This application claims the benefit under 35 U.S.C. Section 119(e) of co-pending and commonly-assigned U.S. Provisional Patent Application Ser. No. 63/104,154, filed on Oct. 22, 2020, and entitled “MONOAMINE OXIDASE BLOCKADE THERAPY FOR TREATING CANCER THROUGH REGULATING ANTITUMOR T CELL IMMUNITY” which application is incorporated by reference herein.

### STATEMENT REGARDING FEDERALLY SPONSORED RESEARCH AND DEVELOPMENT

This invention was made with government support under Grant Number CA196335, awarded by the National Institutes of Health. The government has certain rights in the invention.

### TECHNICAL FIELD

The present invention relates to methods and materials for treating cancers.

### BACKGROUND OF THE INVENTION

CD8 cytotoxic T cells are potent immune cells capable of recognizing and eradicating malignant cells; these immune cells are therefore attractive therapeutic targets for treating cancer (1-3). However, the antitumor responses of CD8 T cells can be severely restrained by negative regulator (immune checkpoint) pathways that are particularly prevalent in the tumor immunosuppressive environment (4). To release this suppression and harness the antitumor potential of CD8 T cells, several immune checkpoint blockade (ICB) therapies have been developed over the past decade (5, 6). Notably, blockade of the CTLA-4 and PD-1/PD-L1 inhibitory pathways have achieved remarkable clinical responses and revolutionized the treatment of many cancers; so far FDA has approved these two ICB therapies for treating more than 10 different malignancies (5, 6). Despite these impressive successes, only a fraction of cancer patients respond to CTLA-4 and PD-1/PD-L1 blockade therapies, and most responders suffer tumor recurrence due to the development of tumor immune evasion (7). These limitations of existing ICB therapies are thought to be largely caused by the presence of multiple immune checkpoint pathways, as well as the different roles of individual immune checkpoint pathways in regulating specific cancer types and disease stages (7).

For the reasons noted above, there is a need in the art the identification of new immune checkpoints and the associated development of materials and methods for new combination treatments in cancer immunotherapies.

### SUMMARY OF THE INVENTION

Monoamine oxidase A (MAO-A) is an enzyme best known for its function in the brain, where it breaks down neurotransmitters and thereby influences mood and behavior. Small molecule MAO inhibitors (MAOIs) are clinically used for treating depression and other neurological disorders. However, the involvement of MAO-A in antitumor immunity has not been known.

Here we report the discovery of the induction of the Maoa gene in tumor-infiltrating immune cells. Moreover, Maoa knockout mice were observed to exhibit enhanced antitumor T cell immunity and suppressed tumor growth. Harnessing these discoveries, we then determined that MAOI treatment significantly suppressed tumor growth in preclinical mouse syngeneic and human xenograft tumor models in a T cell-dependent manner. Unexpectedly, we then discovered that combining MAOI and anti-PD-1 treatments generated synergistic tumor suppression effects. In addition, clinical data correlation studies associated intratumoral MAOA expression with T cell dysfunction and impaired patient survival in a broad range of cancers. We further demonstrated that MAO-A restrains antitumor T cell immunity through controlling intratumoral T cell autocrine serotonin signaling. Together, these data identify MAO-A as an immune checkpoint. Building upon this discovery, we have developed materials and methods for the use of MAOI antidepressants in cancer immunotherapies.

The invention disclosed herein has a number of embodiments. Embodiments of the invention include compositions of matter comprising a chemotherapeutic agent and a monoamine oxidase A inhibitor (and optionally a pharmaceutically acceptable carrier). Typically in these embodiments, a monoamine oxidase A inhibitor is present in the composition in such that amounts of monoamine oxidase A inhibitor available for CD8 T cells in an individual administered the composition are sufficient to modulate the phenotype of the CD8 T cells (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like).

In certain embodiments of the invention, a monoamine oxidase A inhibitor in the composition comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone. Optionally, the monoamine oxidase A inhibitor is disposed within a nanoparticle; for example a nanoparticle comprising a lipid or the like. The compositions of the invention can include a variety of different chemotherapeutic agents. Optionally for example, a composition of the invention includes at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

Another embodiment of the invention is a method of modulating a phenotype of a tumor-infiltrating CD8 T cell comprising introducing a monoamine oxidase A inhibitor in the environment in which the CD8 T cell is disposed; wherein amounts of the monoamine oxidase A inhibitor introduced into the environment are selected to be sufficient to modulate the phenotype of the tumor-infiltrating CD8 T cell (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like, as compared to control cells not exposed to the monoamine oxidase A inhibitor). In certain embodiments of the invention, the tumor-infiltrating CD8 T cell is disposed in an individual diagnosed with cancer (e.g. a lymphoma or a skin, breast, ovarian, prostate, colorectal or lung cancer), for example a patient undergoing a therapeutic regimen comprising the administration of a chemotherapeutic agent such as an immune checkpoint inhibitor. Typically in these embodiments, modulation of the phenotype of the tumor-infiltrating CD8 T cell comprises at least one of: enhanced tumor immunoreactivity; enhanced secretion of serotonin; increased expression of IFN- [symbol] ; increased expression of Granzyme B; or decreased expression of PD-1. Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. These methods of the invention can introduce a monoamine oxidase A inhibitor into an environment in which CD8 T cells are disposed in combination with a variety of different chemotherapeutic agents. Optionally for example, a method of the invention introduces at least one immune checkpoint inhibitor chemotherapeutic agent, such as one selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

A related embodiment of the invention is a method of treating a cancer (e.g. a lymphoma or a skin, breast, ovarian, prostate, colorectal or lung cancer) in an individual comprising administering to the individual a monoamine oxidase A inhibitor; wherein amounts of the monoamine oxidase A inhibitor administered to the individual are selected to be sufficient to modulate the phenotype of tumor-infiltrating CD8 T cells in the individual (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like). Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. In certain embodiments, the individual is undergoing a therapeutic regimen comprising the administration of at least one chemotherapeutic agent, such as one selected to affect a CTLA-4 or a PD-1/PD-L1 blockade. Some embodiments of the invention include methods of administering monoamine oxidase A inhibitor to the individual in combination with a chemotherapeutic agent. Optionally for example, a method of the invention includes administering a monoamine oxidase A inhibitor to the individual in combination with at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments of the invention, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. MAO-A-deficient mice show suppressed tumor growth and enhanced CD8 T cell antitumor immunity. (A) QPCR analyses of Maoa mRNA expression in tumor-infiltrating immune cells (TIIs) in a mouse B16-OVA melanoma model (n=3). Spleen cells collected from the tumor-bearing and tumor-free B6 mice were included as controls. A. U., artificial unit. (B to D) Syngeneic tumor growth in Maoa-WT and Maoa-KO mice. (B) Experimental design. (C) MC38 colon cancer tumor growth (n=4-5). (D) B16-OVA melanoma tumor growth (n=5). (E to J) Flow cytometry analysis of tumor-infiltrating CD8 T cells in Maoa-WT and Maoa-KO mice carrying B16-OVA tumors (n=4). FACS plots and quantifications are presented showing the measurements of intracellular IFN-γ (E and F) and Granzyme B (G and H) production, as well as cell surface PD-1 expression (I and J). MFI, mean fluorescence intensity. (K to M) scRNAseq analysis of tumor-infiltrating CD8 T cells in Maoa-WT and Maoa-KO mice carrying B16-OVA tumors (10 tumors were combined for each group). (K) t-SNE projection showing the formation of two clusters (C1: resting CD8 T cells; and C2: effector CD8 T cells). Each dot corresponds to one single cell, and is colored according to cell cluster. (L) Heatmap showing the expression of selected genes associated with CD8 T cell activation and functionality. (M) Violin plots showing the expression distribution of Gzmb and Ifng genes. Each dot represents an individual cell. Representative of 1 (K to M), 2 (A), and 3 (B to J) experiments. Data are presented as the mean±SEM. ns, not significant, *P<0.05, **P<0.01, by one-way ANOVA (A) or by Student's t test (C, D, F, H, J). P values of violin plots (M) were determined by Wilcoxon rank sum test.

FIG. 2. MAO-A directly regulates CD8 T cell antitumor immunity. (A to C) Two-way bone marrow (BM) transfer experiments. (A) Experimental design. (B) B16-OVA tumor growth in BoyJ wildtype recipient mice reconstituted with BM cells from Maoa-WT or Maoa-KO donor mice (denoted as BoyJ^{WT-BM} or BoyJ^{KO-BM} mice, respectively) (n=6-7). (C) B16-OVA tumor growth in Maoa-WT or Maoa-KO recipient mice reconstituted with BM cells from BoyJ wildtype donor mice (denoted as WT^{BoyJ-BM} or KO^{BoyJ-BM} mice, respectively) (n=6). (D to F) OT1 T cell adoptive transfer experiment (n=8-10). (D) Experimental design. OT1, OVA-specific transgenic CD8 T cell; OT1-WT, wildtype OT1 T cell; OT1-KO, MAO-A-deficient OT1 T cell. (E) FACS plots showing the detection of intratumoral OT1-WT and OT1-KO T cells (gated as CD45.2⁺CD8⁺ cells). (F) Tumor growth. Representative of 2 experiments. Data are presented as the mean±SEM. ns, not significant, **P<0.01, ***P<0.001, by Student's t test.

FIG. 3. MAO-A acts as a negative-feedback regulator to restrain CD8 T cell activation. (A) QPCR analyses of Maoa mRNA expression in tumor-infiltrating CD8 T cell subsets in a mouse B16-OVA melanoma model (n=3). Naïve CD8 T cells (gated as TCRβ⁺CD8⁺CD44^{lo}CD62L^{hi} cells) sorted from the spleen of tumor-free B6 mice were included as controls. (B to I) Activation of Maoa-WT and Maoa-KO CD8 T cells (n=3-6). CD8 T cells were purified from Maoa-WT and Maoa-KO mice and stimulated in vitro with anti-CD3. The analyses of Maoa mRNA expression (B), cell proliferation (C), activation marker expression (CD25; D and E), effector cytokine production (IL-2 and IFN-γ; F and G), and cytotoxic molecule production (Granzyme B; H and I) are shown, either over a 4-day time course (B, F, G), or at day 3 post anti-CD3 stimulation (C to E, H, I). (J to O) Activation of Maoa-KO CD8 T cells with MAO-A overexpression (n=6). CD8 T cells were isolated from Maoa-KO mice, stimulated in vitro with anti-CD3 and transduced with a MIG-Maoa retrovector or a MIG mock retrovector (J). The analyses of retroviral transduction efficiency (K), Maoa mRNA expression (L), effector cytokine mRNA expression (Il2 and Ifng; M and N), and cytotoxicity molecule mRNA expression (Gzmb; O) at day 4 post-stimulation are presented. Representative of 2 (A, J to O) and 3 (B to I) experiments. Data are presented as the mean±SEM. ns, not significant, *P<0.05, **P<0.01, ***P<0.001, by one-way ANOVA (A) or by Student's t test (B, C, E to G, I, L to O).

FIG. 4. MAO-A regulates CD8 T cell autocrine serotonin signaling. (A) Schematics showing the antigen stimulation-induced serotonin synthesis/degradation loop in a CD8 T cell. Possible pharmacological interventions are indicated. TCR, T cell receptor; 5-HTR, 5-HT (serotonin) receptor; MAOIs, monoamine oxidase inhibitors; ASE, asenapine (an antagonist blocking a majority of 5-HTRs). (B and C) QPCR analyses of Tph1 (B) and Maoa (C) mRNA expression in Maoa-WT and Maoa-KO CD8 T cells, at 24-hour post anti-CD3 stimulation (n=3). (D and E) CD8 T cell serotonin production in vitro (n=3). (D) Serotonin levels in Maoa-WT and Maoa-KO CD8 T cell cultures over a 4-day time course post anti-CD3 stimulation. (E) Serotonin levels in Maoa-WT CD8 T cell cultures at 24-hour post anti-CD3 stimulation, with or without phenelzine treatment (Phe or NT). Serotonin levels were measured by ELISA. (F to K) CD8 T cell effector cytokine production in vitro (n=3). (F and G) IL-2 and IFN-γ levels in Maoa-WT CD8 T cell cultures over a 4-day time course post anti-CD3 stimulation, with or without phenelzine treatment (Phe or NT). (H and I) IL-2 and IFN-γ levels in Maoa-WT CD8 T cell cultures over a 4-day time course post anti-CD3 stimulation, with or without serotonin treatment (SER or NT). (J and K) IL-2 and IFN-γ levels in Maoa-WT and Maoa-KO CD8 T cell cultures at day 2 post anti-CD3 stimulation, with or without asenapine treatment (ASE or NT). Cytokine levels were measured by ELISA. (L and M) CD8 T cell 5-HTR and TCR signaling cross-talk in vitro. Maoa-WT and Maoa-KO CD8 T cells were stimulated with anti-CD3 for 2 days, in the presence or absence of asenapine (ASE). Cells were then rested on ice for 2 hours and restimulated with anti-CD3 for 20 minutes, followed by whole protein extraction (L) or nuclear protein extraction (M), and Western blot analysis of key signaling molecules involved in 5-HTR (L) and TCR (M) signaling pathways. (N and O) CD8 T cell serotonin production in vivo in a B16-OVA melanoma model (n=3). On day 14 post tumor challenge, tumors were collected from experimental mice for serotonin measurement using HPLC. (N) Intatumoral serotonin levels in Maoa-WT and Maoa-KO mice (denoted as WT and KO, respectively). (O) Intratumoral serotonin levels in Maoa-WT mice, with or without phenelzine treatment (Phe or NT), and with or without antibody-induced depletion of CD8 T cells (aCD8 or Iso). Representative of 2 (L to O) and 3 (B to K) experiments. Data are presented as the mean±SEM. ns, not significant, *P<0.05, **P<0.01, ***P<0.001, by two-way ANOVA (B and C) or by Student's t test (D to K, N, O).

FIG. 5. MAO-A blockade for cancer immunotherapy—syngeneic mouse tumor model studies. (A) Effect of MAOI treatment on CD8 T cell activation in vitro. CD8 T cells were purified from B6 wildtype mice and stimulated with anti-CD3 for 3 days, in the absence (NT) or presence of MAOIs (phenelzine, Phe; moclobemide, Moc; and clorgyline, Clo). ELISA analyses of IFN-γ production are presented (n=3). (B and C) Cancer therapy potential of MAOI treatment in a B16-OVA melanoma model. (B) Experimental design. B6 wildtype mice were untreated (NT) or treated with MAOIs (Phe, Moc, and Clo). (C) Tumor growth (n=6-8). (D to F) Cancer therapy potential of MAOI treatment and its CD8 T cell-dependency in a B16-OVA melanoma model. (D) Experimental design. (E) Tumor growth in B6 wildtype mice with or without phenelzine treatment (Phe or NT; n=7-9). (F) Tumor growth in B6 wildtype mice with or without phenelzine treatment and with or without anti-CD8 treatment to deplete CD8 T cells (Iso, Phe+Iso, or Phe+αCD8; n=6-9). (G to I) Cancer therapy potential of MAOI treatment in combination with anti-PD-1 treatment in a MC38 colon cancer model and a B16-OVA melanoma model. (G) Experimental design. B6 wildtype mice were inoculated with tumor cells, with or without phenelzine treatment and with or without anti-PD-1 treatment (Iso, Phe+Iso, αPD-1, or Phe+αPD-1). (H) MC38 tumor growth (n=5). (I) B16-OVA tumor growth (n=4-5). Representative of 2 (B to I) and 3 (A) experiments. Data are presented as the mean±SEM. ns, not significant, *P<0.05, **P<0.01, ***P<0.001, by one-way ANOVA (A, C, F, H, I) or by Student's t test (E).

FIG. 6. MAO-A blockade for cancer immunotherapy—human T cell and clinical data correlation studies. (A to D) Translational potential of MAOI treatment for improving human CD8 T cell antitumor reactivity. (A) QPCR analyses of MAOA mRNA expression in naïve and anti-CD3/anti-CD28 stimulated human CD8 T cells of random healthy donors (n=5). (B) Schematics showing a human tumor-T cell pair designated for the study of human CD8 T cell antitumor reactivity. A375-A2-ESO-FG, a human A375 melanoma cell line engineered to express an NY-ESO-1 tumor antigen, its matching MHC molecule (HLA-A2), and a dual reporter comprising a firefly luciferase and an enhanced green fluorescence protein (FG). ESO-T, human peripheral blood CD8 T cells engineered to express an NY-ESO-1 antigen-specific TCR (ESO-TCR; clone 3A1). ESOp, NY-ESO-1 peptide. (C) Experimental design to study the cancer therapy potential of MAOI treatment in a human T cell adoptive transfer and human melanoma xenograft NSG mouse model. NT, no treatment; Phe, phenelzine treatment. (D) Tumor growth from C (n=9-10). (E and F) Clinical data correlation studies. A Tumor Immune Dysfunction and Exclusion (TIDE) computational method was used to study the association between the tumor-infiltrating CD8 T cell (cytotoxic T lymphocyte, CTL) level and overall patient survival in relation to the intratumoral MAOA gene expression level. For each patient cohort, tumor samples were divided into MAOA-high (samples with MAOA expression one standard deviation above the average; shown in left survival plot) and MAOA-low (remaining samples; shown in right survival plot) groups, followed by analyzing the association between the CTL levels and survival outcomes in each group. The CTL level was estimated as the average expression level of CD8A, CD8B, GZMA, GZMB, and PRF1. Each survival plot presented tumors in two subgroups: “CTL-high” group (red) had above-average CTL values among all samples, while ‘CTL-low’ group (blue) had below-average CTL values. A T cell dysfunction score (z score) was calculated for each patient cohort, correlating the MAOA expression level with the beneficial effect of CTL infiltration on patient survival. A positive z score indicates that the expression of MAOA is negatively correlated with the beneficial effect of tumor-infiltrating CTL on patient survival. The P value indicates the comparison between the MAOA-low and MAOA-high groups, and was calculated by two-sided Wald test in a Cox-PH regression. (E) TIDE analysis of a colon cancer patient cohort (GSE29621; n=65). z=2.31; P=0.0208. (F) TIDE analysis of a melanoma patient cohort receiving anti-PD-1 treatment (ENA PRJEB23709; n=41). z=2.16; P=0.0305. Representative of 2 experiments. Data are presented as the mean±SEM. ***P<0.001, by Student's t test (A and D).

FIG. 7. Characterization of Maoa-KO mice, related to FIG. 1. (A and B) MAO-A expression in the lymphoid organs of Maoa-WT and Maoa-KO mice (n=3). (A) Western blot analyses of MAO-A protein expression. (B) QPCR analyses of Maoa mRNA expression. ND, nondetectable. (C to E) T cell development in the thymi of Maoa-WT and Maoa-KO mice (n=3). (C) FACS plots showing the developmental stages of thymocytes defined by CD4/CD8 co-receptor expression. DN, double-negative; DP, double-positive; CD4 SP, CD4 single-positive; CD8 SP, CD8 single-positive. (D) Quantification of C. (E) Numbers of total thymocytes. (F to H) T cell presence in the periphery of Maoa-WT and Maoa-KO mice (n=3). (F) FACS plots showing the detection of CD4 and CD8 T cells (gated as TCRβ⁺CD4⁺ and TCRβ⁺CD8⁺ cells, respectively) in the peripheral blood, spleen, and lymph nodes. (G) Quantification of F. (H) Numbers of total splenocytes. (I) T cell phenotype in the spleen of Maoa-WT and Maoa-KO mice prior to tumor challenge (n=3). Representative FACS plots are presented, showing a typical naïve phenotype (CD25^{lo}CD69^{lo}CD44^{lo}CD62L^{hi}) of T cells in both mice. Representative of 2 experiments. Data are presented as the mean±SEM. ns, not significant, ***P<0.001, by Student's t test.

FIG. 8. MAO-A-deficient mice show suppressed tumor growth and enhanced CD8 T cell antitumor immunity, related to FIG. 1. (A) T cell tumor infiltration in Maoa-WT and Maoa-KO mice bearing B16-OVA tumors (n=5). Representative FACS plots are presented, showing the detection of comparable levels of tumor-infiltrating immune cells (TIIs; pre-gated as CD45.2⁺ cells) comprising CD4 and CD8 T cells (gated as TCRβ⁺CD4⁺ and TCRβ⁺ CD8⁺ cells, respectively). (B) The gene expression profiles of tumor-infiltrating CD8 T cells in Maoa-WT and Maoa-KO mice bearing B16-OVA tumors measured by scRNASeq (10 tumors were combined for each group). Heatmap is presented, showing the expression of selected genes associated with T cell activation and functionality. Each column indicates a single cell. Each row indicates a selected gene. Representative of 1 (B) and 3 (A) experiments.

FIG. 9. MAO-A directly regulates antitumor immunity, related to FIG. 2, A-C. (A and B) T cell reconstitution in BoyJ wildtype recipient mice receiving bone marrow (BM) cells from the Maoa-WT or Maoa-KO donor mice (denoted as BoyJ^{WT-BM} or BoyJ^{KO-BM} mice, respectively; n=6-7). (A) FACS plots showing the detection of comparable levels of CD4 and CD8 T cells (gated as CD45.2⁺CD4⁺ and CD45.2⁺CD8⁺ cells, respectively) in the blood of BoyJ^{WT-BM} or BoyJ^{KO-BM} mice. (B) Quantification of A. (C and D) T cell reconstitution in Maoa-WT and Maoa-KO recipient mice receiving bone marrow cells from BoyJ wildtype donor mice (denoted as WT^{BoyJ-BM} or KO^{BoyJ-BM} mice, respectively; n=6). (C) FACS plots showing the detection of comparable levels of CD4 and CD8 T cells (gated as CD45.2⁻CD4⁺ and CD45.2⁻CD8⁺ cells, respectively) in the blood of WT^{BoyJ-BM} or KO^{BoyJ-BM} mice. (D) Quantification of C. Representative of 2 experiments. Data are presented as the mean±SEM. ns, not significant, by Student's t test.

FIG. 10. MAO-A directly regulates CD8 T cell antitumor immunity, related to main FIG. 2, D-F. (A) Breeding strategy for the generation of OT1 transgenic (OT1-Tg) mice deficient of Maoa gene (denoted as the OT1-Tg/Maoa-KO mice). (B) FACS plots showing the isolation of high-purity OT1 transgenic T cells (>99% purity; gated as CD4⁻CD8⁺TCR Vβ5⁺ cells) from the OT1-Tg and OT1-Tg/Maoa-KO mice (denoted as OT1-WT and OT1-KO T cells, respectively). (C to H) Antitumor reactivity of OT1-WT and OT1-KO T cells adoptively transferred into BoyJ mice bearing B16-OVA tumors (n=8-10). The experimental design is shown in FIG. 2D. On day 17, tumors were collected from experimental mice, followed by TII isolation and FACS analysis. (C) FACS quantification of tumor-infiltrating OT1-WT and OT1-KO T cells (gated as CD45.2⁺CD8⁺ cells). (D) FACS plots showing the antigen-experienced phenotype (CD44^{hi}CD62L^{lo}) of tumor-infiltrating OT1-WT and OT1-KO T cells. Note prior to adoptive transfer, both the purified OT1-WT and OT1-KO T cells displayed a naïve T cell phenotype (CD44^{lo}CD62^{hi}). (E) FACS plot showing the PD-1 expression on tumor-infiltrating OT1-WT and OT1-KO T cells. (F) Quantification of E. (G) FACS plots showing the measurements of intracellular cytokine (IFN-γ and TNF-α production by tumor-infiltrating OT1-WT and OT1-KO T cells. (H) Quantification of G. Representative of 2 experiments. Data are presented as the mean±SEM. ns, not significant; *P<0.05, **P<0.01, by Student's t test.

FIG. 11. MAO-A acts as a negative-feedback regulator to restrain CD8 T cell activation; studying antigen-specific T cells, related to FIG. 3. OVA-specific OT1 transgenic CD8 T cells were isolated from the OT1-Tg or OT1-Tg/Maoa-KO mice (denoted as OT1-WT or OT1-KO T cells, respectively), then stimulated in vitro with anti-CD3 (n=3). (A) FACS plot showing the CD25 expression on OT1-WT and OT1-KO T cells at day 3 post anti-CD3 stimulation. (B) Quantification of A. (C and D) ELISA analyses of IL-2 (C) and IFN-γ (D) production by OT1-WT and OT1-KO T cells over a 4-day time course post anti-CD3 stimulation. Representative of 3 experiments. Data are presented as the mean±SEM. *P<0.05, **P<0.01, by Student's t test.

FIG. 12. MAO-A regulates intratumoral CD8 T cell autocrine serotonin signaling, related to FIG. 4, N-O. (A) Schematic model showing the crosstalk between 5-HTR and TCR signaling pathways in a CD8 T cell. Experimentally confirmed signaling molecules and pathways are highlighted in red. (B to D) CD8 T cell serotonin production in vivo in a B16-OVA melanoma model (n=3). On day 14 post tumor challenge, sera were collected from experimental mice for serotonin measurement using HPLC. (B) Serum serotonin levels in Maoa-WT and Maoa-KO mice (denoted as WT and KO, respectively). (C) Representative FACS plots showing the successful in vivo depletion of CD8 T cells induced by anti-CD8 antibody injection. (D) Serum serotonin levels in Maoa-WT mice, with or without phenelzine treatment (Phe or NT), and with or without antibody-induced depletion of CD8 T cells (aCD8 or Iso). Representative of 2 experiments. Data are presented as the mean SEM. ns, not significant, by Student's t test.

FIG. 13. MAOI treatment induces CD8 T cell hyperactivation in vitro, related to FIG. 5A. (A) Experimental design. CD8 T cells were purified from B6 wildtype mice and stimulated with anti-CD3 in vitro, in the absence (NT) or presence of MAOIs (phenelzine, Phe; moclobemide, Moc; and clorgyline, Clo). N=3. (B) FACS plots showing the measurement of cell surface CD25 expression on day 2 post anti-CD3 stimulation. (C) Quantification of (B). (D) FACS plots showing the measurement of intracellular Granzyme B production on day 2 post anti-CD3 stimulation. (E) Quantification of (D). (F) ELISA analyses of IL-2 production on day 3 post anti-CD3 stimulation. Representative of 3 experiments. Data are presented as the mean±SEM. *P<0.05, **P<0.01, ***P<0.001, by one-way ANOVA.

FIG. 14. MAO-A blockade for cancer immunotherapy—syngeneic mouse tumor model studies, related to FIG. 5. (A to F) Studying phenelzine treatment in a B16-OVA melanoma model (n=5-7). B6 wildtype mice were inoculated with B16-OVA melanoma cells, with or without phenelzine treatment (Phe or NT). On day 17, tumors were collected, followed by TII isolation and FACS analysis. The analyses of tumor-infiltrating CD8 T cells (pre-gated as CD45.2⁺TCRβ⁺CD8⁺ cells) for their intracellular production of effector cytokines (i.e., IFN-γ; A and B) and cytotoxic molecules (i.e., Granzyme B; C and D), and their surface expression of exhaustion markers (i.e., PD-1; E and F) are presented. (G and H) Studying MC38 tumor growth in immunodeficient NSG mice with or without phenelzine treatment (Phe or NT). (G) Experimental design. (H) Tumor growth (n=5-6). (I and J) Studying B16-OVA tumor growth in NSG mice with or without phenelzine treatment (Phe or NT). (I) Experimental design. (J) Tumor growth (n=3-4). Representative of 2 (G to J) and 3 (A to F) experiments. Data are presented as the mean±SEM. ns, not significant, *P<0.05, **P<0.01, by Student's t test.

FIG. 15. MAO-A blockade for cancer immunotherapy—human T cell studies, related to FIG. 6, A-D. (A) Schematics showing the strategy to generate human CD8 T cells recognizing the NY-ESO-1 tumor antigen. Healthy donor peripheral blood mononuclear cells (PBMCs) were stimulated in vitro with anti-CD3/CD28 and IL-2 to expand human CD8 T cells, followed by transduction with a Retro/ESO-TCR retrovector encoding an HLA-A2-restricted NY-ESO-1 specific TCR (clone 3A1). The resulting human CD8 T cells, denoted as the ESO-T cells, can specifically target the A375-A2-ESO-FG human melanoma cells. (B) FACS plots showing the detection of ESO-TCR expression on the engineered ESO-T cells. Human CD8 T cells that received mock transduction were included as a staining control (denoted as Mock-T cells). (C) Experimental design to study the in vitro direct killing of A375-A2-ESO-FG human melanoma cells by ESO-T cells. (D) Tumor killing data from C (n=3-6). (E and F) A375-A2-ESO-FG tumors growth in NSG mice with or without phenelzine treatment (Phe or NT). (E) Experimental design. (F) Tumor growth (n=6). Representative of 2 experiments. Data are presented as the mean±SEM. ns, not significant, *P<0.05, ***P<0.001, by one-way ANOVA (D) or by Student's t test (F).

FIG. 16. Clinical data correlation studies identify MAO-A as a negative regulator of T cell antitumor function in cancer patients, related to FIG. 6, E-F. A Tumor Immune Dysfunction and Exclusion (TIDE) computational method was used to study the association between the tumor-infiltrating CD8 T cell (cytotoxic T lymphocyte, CTL) level and overall patient survival in relation to the intratumoral MAOA gene expression level. For each patient cohort, tumor samples were divided into MAOA-high (samples with MAOA expression one standard deviation above the average; shown in left survival plot) and MAOA-low (remaining samples; shown in right survival plot) groups, followed by analyzing the association between CTL levels and survival outcomes in each group. The CTL level was estimated as the average expression level of CD8A, CD8B, GZMA, GZMB, and PRF1. Each survival plot presented tumors in two subgroups: “CTL-high” group (red) had above-average CTL values among all samples, while ‘CTL-low’ group (blue) had below-average CTL values. A T cell dysfunction score (z score) was calculated for each patient cohort, correlating the MAOA expression level with the beneficial effect of CTL infiltration on patient survival. A positive z score indicates that the expression of MAOA is negatively correlated with the beneficial effect of tumor-infiltrating CTL on patient survival. The P value indicates the comparison between the MAOA-low and MAOA-high groups, and was calculated by two-sided Wald test in a Cox-PH regression. (A) TIDE analysis of a lung cancer patient cohort (GSE30219; n=263). z=2.56; P=0.0104. (B) TIDE analysis of a cervical cancer patient cohort (TCGA; n=296). z=2.28; P=0.0224. (C) TIDE analysis of a pancreatic cancer patient cohort (GSE21501@PRECOG; n=102). z=2.16; P=0.0305.

FIG. 17. The “intratumoral MAO-A-serotonin axis” model. (A) Schematics showing the “MAO-A-serotonin axis” in the brain regulating neuron activity. A presynaptic neuron and a postsynaptic neuron form a neuron synapse. The presynaptic neuron produces serotonin, which is provided to the postsynaptic neuron for neuronal signal transmission. The presynaptic neuron also expresses MAO-A that controls serotonin degradation thereby regulating neuron activity. MAOIs have been clinically used for treating depression symptoms, targeting the “MAO-A-serotonin axis” in the brain. (B) Schematics showing the “MAO-A-serotonin axis” in tumors regulating CD8 T cell antitumor reactivity. Analogous to neurons in brain, a tumor-specific CD8 T cell and a tumor cell form an immune synapse. The CD8 T cell produces serotonin that enhances TCR/TA (tumor antigen) recognition-induced T cell activation. The CD8 T cell also expresses MAO-A, which controls serotonin degradation thereby regulating CD8 T cell antitumor reactivity. Established MAOI antidepressants can potentially be repurposed for enhancing T cell-based cancer immunotherapy, targeting the “MAO-A-serotonin axis” in tumors.

FIG. 18. Delivery of phenelzine using cMLV. (A) Schematics of cMLV. (B-C). Study the cancer therapy potential of cMLV-formulated phenelzine (cMLV-Phe, 30 mg/kg) in a B16-OVA mouse melanoma model. Free phenelzine (Free-Phe, 30 mg/kg) was included as a control. (B) Experimental design. (C) Tumor growth (n=5). Data are presented as the mean±SEM. ns, not significant, ***P<0.001, ****P<0.0001, by one-way ANOVA.

FIG. 19. Behavioral study of B16-OVA tumor-bearing mice treated with either free or cMLV-formulated phenelzine (Free-Phe or cMLV-Phe; i.v., q3d). N=5-6. (A) Percentage of animals showing medium to strong aggression. (B) Quantification of aggression bouts per trial across different conditions. (C) Quantification of latency to the onset of aggression in each trial across different conditions. (D) Quantification of total time the animals engage in aggressive behavior in each trial across different conditions. (E) Representative raster plots showing aggression. (F) Phenelzine (Phe) measurements in the brain (n=3). Data are presented as the mean±SEM. ***P<0.001, by one-way ANOVA.

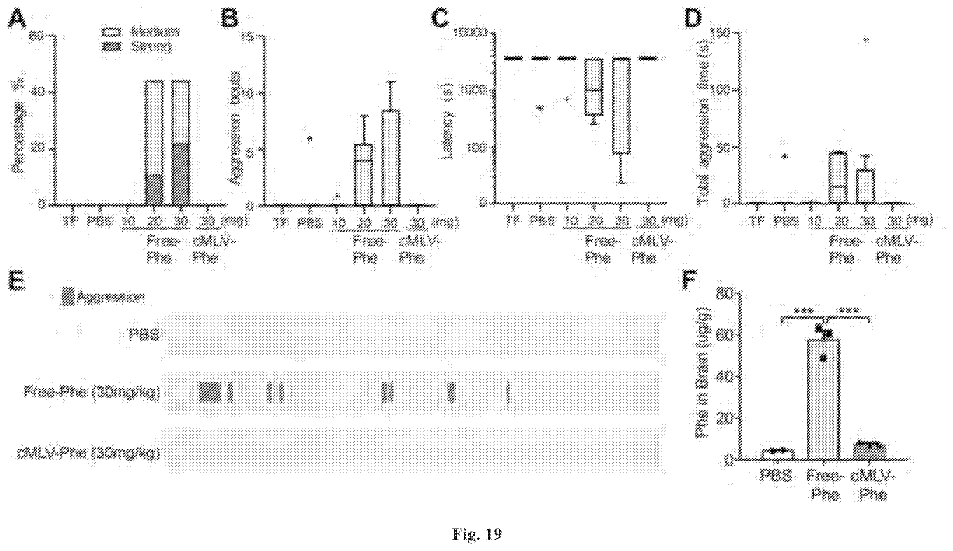

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Monoamine oxidase A (MAO-A) is an enzyme that catalyzes the degradation of biogenic and dietary monoamines (8, 9). MAO-A is located on the outer membrane of mitochondria and in humans is encoded by the X-linked MAOA gene. MAO-A is best known for its function in the brain, where it regulates the homeostasis of key monoamine neuronal transmitters including serotonin, dopamine, epinephrine, and norepinephrine, and thereby influences human mood and behavior (8, 9). Complete MAO-A deficiency in humans caused by a mutation of the MAOA gene leads to an excess of monoamine neuronal transmitters in the brain and results in Brunner syndrome, which is characterized by problematic impulsive behaviors and mood swings (10). Genetic association studies also identified several MAOA gene variants linked to altered MAO-A enzyme expression levels: low-activity forms of the MAOA gene are associated with aggression and hyperactivity disorders; while high-activity forms are associated with depression disorders (11, 12). Notably, due to its link with aggressive and even violent behavior in men, a low-activity variant of the MAOA gene, MAOA-L, has previously received broad publicity and is popularly referred to as the “warrior gene” (13). On the other hand, small molecule MAO inhibitors (MAOIs) have been developed and are clinically utilized for treating depression symptoms (14). However, MAO-A's functions outside of the brain are largely unexplored. In particular, the involvement of MAO-A in antitumor immunity is unknown. In this report, we investigated the role of MAO-A in regulating CD8 T cell antitumor immunity and evaluated the possibility of repurposing MAOIs for cancer immunotherapy, using knockout and transgenic mice, preclinical mouse syngeneic and human xenograft tumor models, and clinical data correlation studies.

As discussed in detail below, we report the discovery of the induction of the Maoa gene in tumor-infiltrating immune cells. Moreover, Maoa knockout mice were observed to exhibit enhanced antitumor T cell immunity and suppressed tumor growth. Harnessing these discoveries, we then determined that MAOI treatment significantly suppressed tumor growth in preclinical mouse syngeneic and human xenograft tumor models in a T cell-dependent manner. Unexpectedly, we then discovered that combining MAOI and anti-PD-1 treatments generated synergistic tumor suppression effects. In addition, clinical data correlation studies associated intratumoral MAOA expression with T cell dysfunction and impaired patient survival in a broad range of cancers. We further demonstrated that MAO-A restrains antitumor T cell immunity through controlling intratumoral T cell autocrine serotonin signaling. Together, these data identify MAO-A as an immune checkpoint and provide strong evidence for the use of MAOI antidepressants in cancer immunotherapies. Embodiments of the invention are based upon these discoveries.

The invention disclosed herein has a number of embodiments. Embodiments of the invention include compositions of matter comprising a chemotherapeutic agent; a monoamine oxidase A inhibitor; and optionally a pharmaceutically acceptable carrier. Typically in these embodiments, a monoamine oxidase A inhibitor is present in the composition in such that amounts of monoamine oxidase A inhibitor available for CD8 T cells in an individual administered the composition are sufficient to modulate the phenotype of the CD8 T cells (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like).

In certain embodiments of the invention, a monoamine oxidase A inhibitor in the composition comprises at least one of: phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone. Optionally, the monoamine oxidase A inhibitor is disposed within a nanoparticle; for example a nanoparticle comprising a lipid or the like. In particular, embodiments of the invention can utilize such nanocarriers to address the short circulatory half-life of free MAOI; limited cancer targeting/penetration; and toxicity of MAOI in CNS. Illustrative nanocarriers include lipid-coated mesoporous silica nanoparticles (“silicasomes”) as well as liposome platforms. In certain embodiments, the nanocarrier is designed to have a size, a charge, one or more surface coatings (e.g., PEG, copolymers), one or more targeting ligands (e.g., peptides) and the like; an optionally the inclusion of imaging agents and the like, with a view to obtaining colloidal stability, low opsonization, long circulatory t½, and effective biodistribution post intravenous (IV) injection.

One such nanocarrier embodiment comprises the irreversible, non-selective MAOI phenelzine because its chemical properties (water solubility of 11.1 mg/mL, Log P 1.2 and pKa 5.5). Other possible MAOIs that are suitable for loading include isocarboxazid and tranylcypromine. Liposomes can be synthesized using lipid biofilm, rehydration, sonication and extrusion (e.g. using membrane of 100 nm pore size) protocols. One can, for example, use a lipid bilayer that exhibits an DSPC/Cholesterol/DSPE-PEG2000 at molar ratio 3:2:0.15. For silicasome embodiments, a bare MSNP core can be constructed using a templating agent and silica precursors to make 80-90 nm particles. The particles can be produced in big batch sizes (e.g., ˜5 g/batch) and stably stored for 18-24 months, allowing aliquots to be removed at different project stages for carrier development. Phenelzine can be remotely imported using different trapping agents, such as triethylammmonium sucrose octasulfate, (NH4)2SO4 or citric acid. Lipid coatings can be introduced using ethanol injection method with controlled sonication power.

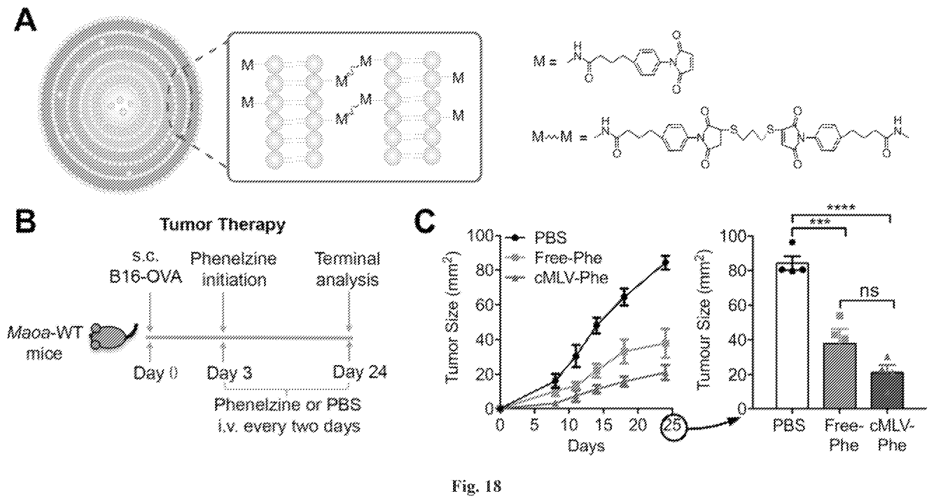

In certain embodiments of the invention, the monoamine oxidase A inhibitor is disposed within a composition comprising a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and the monoamine oxidase A inhibitor disposed within the liposome (see, e.g. FIG. 18). Such liposome compositions are known in the art and discussed, for example, in: U.S. Patent Application Publication No. 20140356414; Joo et al. Biomaterials 34, 3098-3109, doi: 10.1016/j.biomaterials.2013.01.039 (2013); Liu et al., Biomed Res Int 2013, 378380, doi:10.1155/2013/378380 (2013); Liu et al., Mol Pharm 11, 1651-1661, doi:10.1021/mp5000373 (2014); Liu et al., PLoS One 9, e110611, doi:10.1371/journal.pone.0110611 (2014); Kim, Y. J. et al. Mol Pharm 12, 2811-2822, doi:10.1021/mp500754r (2015); and Zhang, X. et al. RSC Adv 7, 19685-19693, doi:10.1039/c7ra01100h (2017), the contents of which are incorporated herein by reference.

Based on the growing awareness that tumor targeting and/or the activation of tumor transcytosis mechanism may generate more robust access in multiple solid tumors, we can make nanoparticles having targeting agents by introducing peptide conjugation to the LB (e.g. iRGD and tumor targeting Arg-Gly-Asp peptide), using a thiol-maleimide reaction to link the cysteine-modified peptide to DSPE-PEG2000-maleimide. All the MAOI nanocarriers can be thoroughly characterized for physicochemical properties, such as size, morphology (cryoEM), loading capacity, release profile, zeta potential, impurities, and stability in biological fluids before use. The biological activity of nMAOIs can be read out using a pre-established in vitro mouse T cell activation assay, by measuring nMAOI regulation of T cell proliferation and IFN-γ/Granzyme B production.

In certain embodiments of the invention, the monoamine oxidase A inhibitor is present in the composition in specific amounts such as at least 100 mg, or at least 250 mg, or at least 500 mg (e.g. of moclobemide). However, in view of the fact that different people weigh different amounts and may respond differently to a specific amount of a monoamine oxidase A inhibitor, those of skill in this art understand that a more precise way to describe embodiments of the invention is to include a description of what the composition does (e.g. enhances tumor immunoreactivity; enhances secretion of serotonin; increases expression of IFN- [symbol] ; increases expression of Granzyme B; decreases expression of PD-1 or the like), rather than by what the composition is (e.g. 100 mg of a monoamine oxidase A inhibitor). In view of the well studied pharmacology of monoamine oxidase A inhibitors, the disclosure provided herein along with the known pharmacodynamics of monoamine oxidase A inhibitors (see, e.g. Holford et al; Br J Clin Pharmacol. 1994 May; 37(5):433-9 for moclobemide) makes the dosing associated with a desired effect to be routine in the art.

The compositions of the invention can include a variety of different chemotherapeutic agents. Optionally for example, a composition of the invention includes at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

The compositions of the invention comprising monoamine oxidase A inhibitor may be made and then systemically administered in combination with a pharmaceutically acceptable vehicle such as an inert diluent. For oral therapeutic administration, the compounds may be combined with one or more excipients and used in the form of ingestible tablets, buccal tablets, troches, capsules, elixirs, suspensions, syrups, wafers, and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's). Common illustrative excipients include antimicrobial agents and buffering agents.

The compositions of the invention comprising monoamine oxidase A inhibitor may be administered parenterally, such as intravenously or intraperitoneally by infusion or injection. Solutions of the compositions of the invention comprising monoamine oxidase A inhibitor can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

Another embodiment of the invention is a method of modulating a phenotype of a tumor-infiltrating CD8 T cell comprising introducing a monoamine oxidase A inhibitor in the environment in which the CD8 T cell is disposed; wherein amounts of the monoamine oxidase A inhibitor introduced into the environment are selected to be sufficient to modulate the phenotype of the tumor-infiltrating CD8 T cell (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like so that the phenotype is modulated). In certain embodiments of the invention, the tumor-infiltrating CD8 T cell is disposed in an individual diagnosed with cancer (e.g. a lymphoma or a skin, breast, ovarian, prostate, colorectal or lung cancer), for example a patient undergoing a therapeutic regimen comprising the administration of a chemotherapeutic agent. Typically in these embodiments, modulation of the phenotype of the tumor-infiltrating CD8 T cell comprises at least one of: enhanced tumor immunoreactivity; enhanced secretion of serotonin; increased expression of IFN- [symbol] ; increased expression of Granzyme B; or decreased expression of PD-1. Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. These methods of the invention can introduce a monoamine oxidase A inhibitor into an environment in which CD8 T cells are disposed in combination with a variety of different chemotherapeutic agents. Optionally for example, a method of the invention introduces at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

A related embodiment of the invention is a method of treating a cancer (e.g. a lymphoma or a skin, breast, ovarian, prostate, colorectal or lung cancer) in an individual comprising administering to the individual a monoamine oxidase A inhibitor; wherein amounts of the monoamine oxidase A inhibitor administered to the individual are selected to be sufficient to modulate the phenotype of tumor-infiltrating CD8 T cells in the individual (e.g. wherein modulation of the phenotype comprises enhanced tumor immunoreactivity; enhanced secretion of serotonin; increased expression of IFN- [symbol] ; increased expression of Granzyme B; decreased expression of PD-1 or the like). Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. In certain embodiments, the individual is undergoing a therapeutic regimen comprising the administration of at least one chemotherapeutic agent. Some embodiments of the invention include methods of administering monoamine oxidase A inhibitor to the individual in combination with a chemotherapeutic agent. Optionally for example, a method of the invention includes administering a monoamine oxidase A inhibitor to the individual in combination with at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments of the invention, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

In methods of the invention, the monoamine oxidase inhibitor is administered in a therapeutically effective amount/dose (e.g. an amount sufficient to modulate the phenotype of CD8 T cells in a patient), which may vary depending upon a variety of factors including the specific monoamine oxidase inhibitor; the age, body weight, general health, sex, and diet of the patient; the mode and time of administration; the rate of excretion; the drug combination; the severity of the particular disorder or condition; and the subject undergoing therapy. The pharmacology of monoamine oxidase inhibitors is well known in the art and, using this information in combination with the disclosure presented herein (e.g. the disclosure below and FIG. 18), doses of such inhibitors can be tailored to the individual subject (e.g. in order to modulate the phenotype of CD8 T cells), as is understood and determinable by one skilled in the relevant arts (see, e.g., Monoamine Oxidase Inhibitors: Clinical Pharmacology, Benefits, and Potential Health Risks (Pharmacology—Research, Safety Testing and Regulation) UK ed. Edition by Sushil K. Sharma (Editor); Berkow et al., eds.; Yamada et al., Clinical Pharmacology of MAO Inhibitors: Safety and Future, NeuroToxicology Volume 25, Issues 1-2, January 2004, Pages 215-221; McDaniel et al., Clinical pharmacology of monoamine oxidase inhibitors; Clin Neuropharmacol. 1986; 9(3):207-34. doi: 10.1097/00002826-198606000-00001; Hermann et al., Current place of monoamine oxidase inhibitors in the treatment of depression; CNS Drugs. 2013 October; 27(10):789-9; as well as The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)). The total dose required for each treatment can be administered by multiple doses or in a single dose over the course of a day, or a week or a month, if desired.

Further aspects and embodiments of the invention are discussed in the sections below.

### MAO-A Deficiency Enhances CD8 T Cell Antitumor Immunity

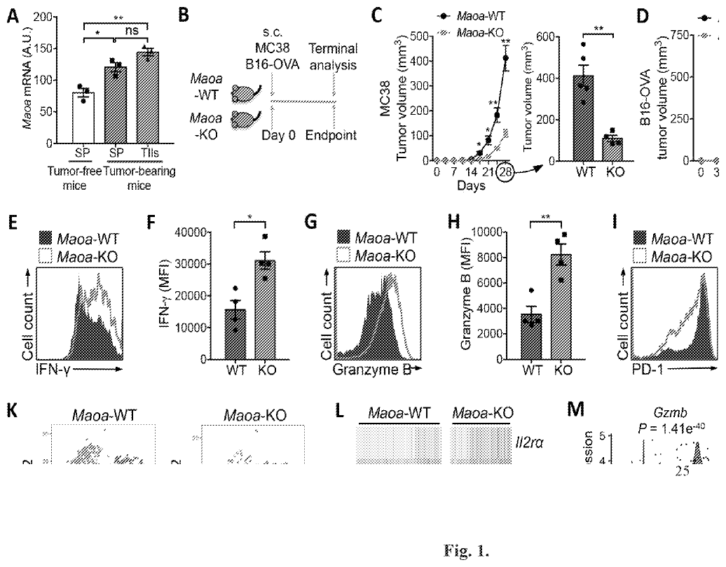

To search for new drug targets regulating antitumor immunity, we grew B16-OVA melanoma solid tumors in C57BL/6J mice, isolated tumor-infiltrating immune cells (TIIs), and evaluated TII gene expression profiles using quantitative RT-PCR. Immune cells isolated from the spleen of tumor-bearing and tumor-free mice were included as controls. In addition to the classical immune regulatory genes, we detected significant changes in the expression of a group of genes typically classified as neuronal regulatory genes. In particular, we detected the induction of a Maoa gene in TIIs (FIG. 1A), suggesting that, along with its known function in the brain as a regulator of neuronal activity (8), it might also function in the tumor as a regulator of antitumor reactivity. We were especially interested to study whether MAO-A might regulate CD8 cytotoxic T cells, which play a critical role in immune response against cancer.

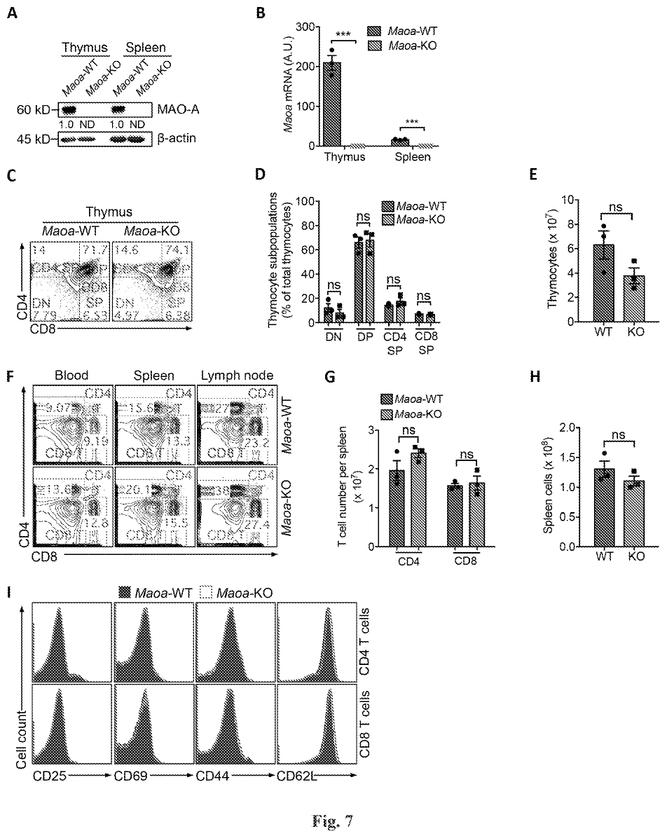

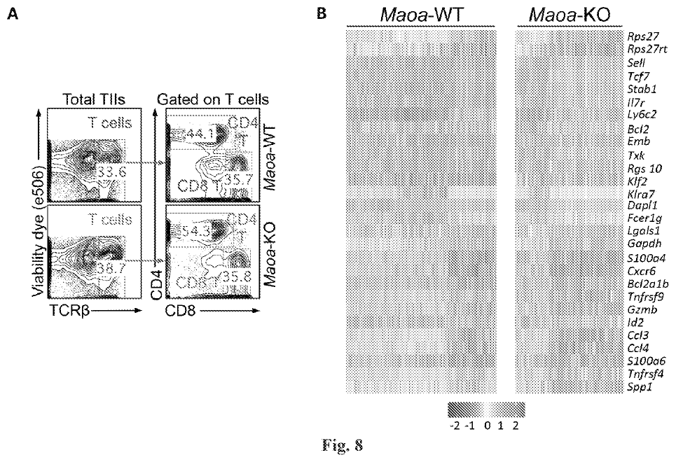

To test this, we began by studying MAO-A-deficient mice that carry a hypomorphic Maoa mutant (15). Although a degree of Maoa expression leakage in the brain was previously reported in these mice (15), analysis of their immune system showed a nearly complete ablation of Maoa mRNA and protein expression in the major immune organs including thymus and spleen (FIGS. 7, A and B). Since we focused on studying immune cells, in this report we refer to these mice as Maoa knockout (Maoa-KO) mice. Maoa-KO mice showed normal T cell development in the thymus and contained normal numbers of T cells in the periphery, compared to the wildtype control mice (Maoa-WT mice) (FIG. 7, C to H). Prior to tumor challenge, these T cells displayed a typical naïve phenotype (CD25^{lo}CD69^{lo}CD44^{lo}CD62L^{hi}; FIG. S1I). When challenged with tumors, compared to Maoa-WT mice, Maoa-KO mice exhibited significantly suppressed tumor growth in two syngeneic mouse tumor models, the MC38 colon cancer model and the B16-OVA melanoma model (FIG. 1, B to D). Flow cytometry analysis detected similar levels of tumor-infiltrating CD8 T cells in Maoa-KO and Maoa-WT mice (FIG. 8A). However, in Maoa-KO mice, these tumor-infiltrating T cells displayed an enhanced effector phenotype: they produced higher levels of effector cytokines and cytotoxic molecules (i.e., IFN-γ and Granzyme B; FIG. 1, E to H), and they expressed lower levels of T cell exhaustion markers (i.e., PD-1; FIG. 1, I and J). Single-cell RNA sequencing (scRNAseq) analysis of tumor-infiltrating CD8 T cells confirmed an enrichment of effector cells in Maoa-KO mice that actively expressed genes associated with cytotoxic T cell activation and functionality (i.e., Il2rα, Gzmb, Prf1, and Ifng; FIG. 1, K to M and FIG. 8B). Therefore, MAO-A is involved in regulating antitumor immunity especially in regulating CD8 T cell antitumor immunity.

### MAO-A Directly Regulates CD8 T Cell Antitumor Immunity

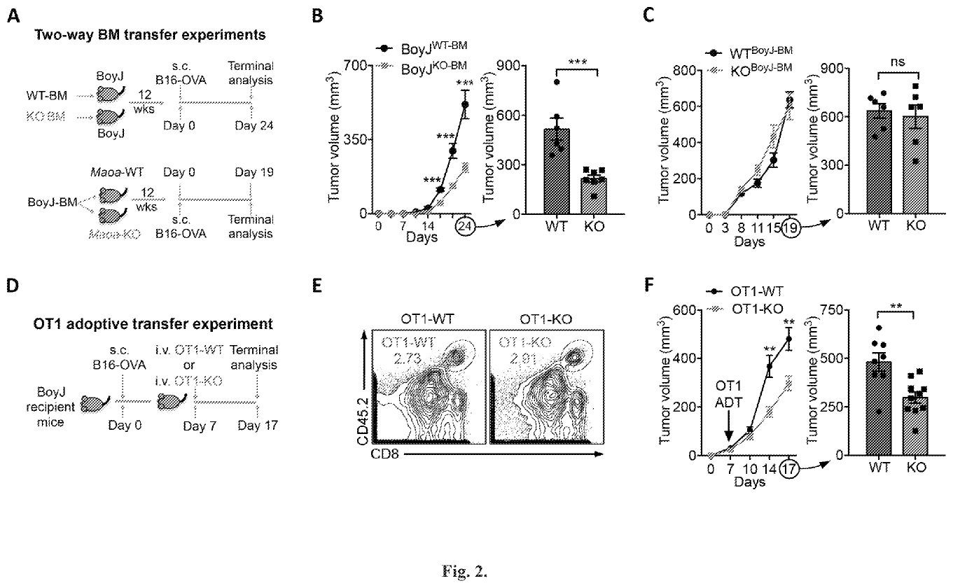

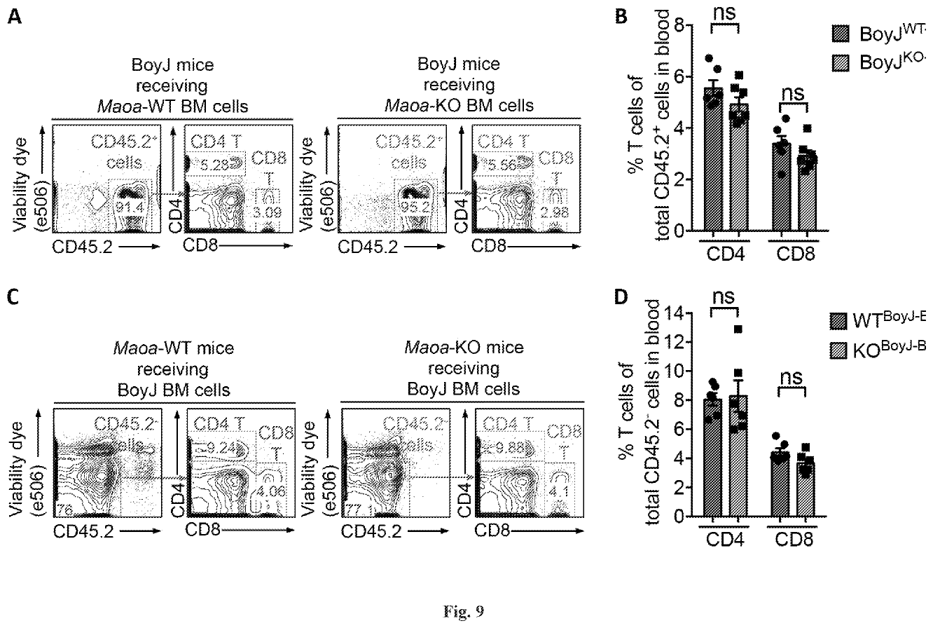

In our Maoa-KO mice tumor challenge study, MAO-A deficiency impacted both immune and non-immune cells (15). To determine whether MAO-A directly or indirectly regulates immune cells, we performed a pair of two-way bone marrow (BM) transfer experiments: in one experiment, we confined MAO-A deficiency comparison to immune cells by reconstituting BoyJ wildtype recipient mice with BM cells from either Maoa-WT or Maoa-KO donor mice followed by B16-OVA tumor challenge; in another experiment, we confined MAO-A deficiency comparison to non-immune cells by reconstituting either Maoa-WT or Maoa-KO recipient mice with BM cells from BoyJ wildtype donor mice followed by B16-OVA tumor challenge (FIG. 2A). Successful reconstitution of immune cells in particular T cells was confirmed in both experiments (FIG. 9, A to D). Suppressed tumor growth was observed only when MAO-A deficiency was confined to the immune cells, indicating that MAO-A affects tumor growth via directly regulating immune cell antitumor reactivity (FIG. 2, B and C).

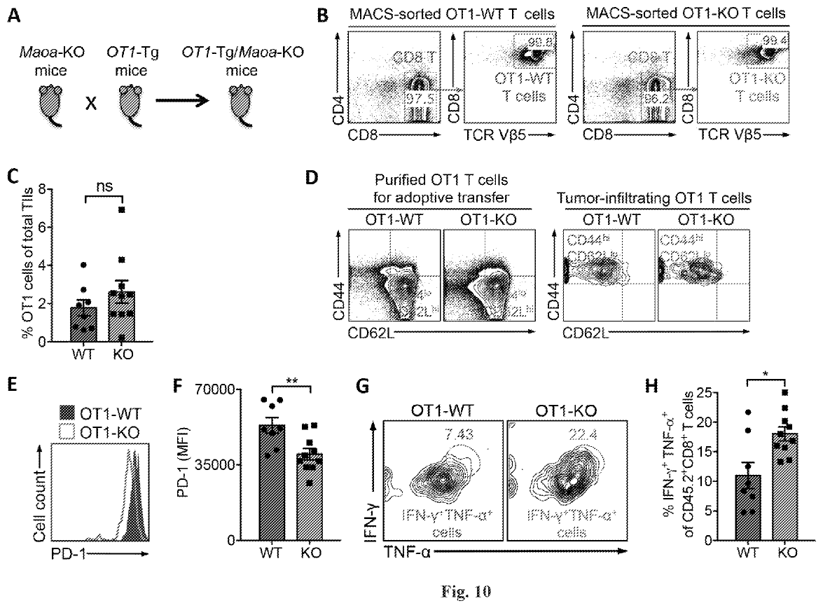

To further study whether MAO-A directly regulates the antitumor reactivity of CD8 T cells, we bred Maoa-KO mice with OT1 transgenic (OT1-Tg) mice and generated OT1-Tg/Maoa-KO mice producing OVA-specific CD8 T cells deficient in MAO-A (FIG. 10A). We isolated OT1 T cells from either the OT1-Tg or OT1-Tg/Maoa-KO mice (denoted as OT1-WT or OT1-KO T cells, respectively) and separately transferred these cells into BoyJ wildtype mice bearing pre-established B16-OVA tumors (FIG. 2D and FIG. 10B). In this experiment, MAO-A deficiency comparison was confined solely to tumor-specific OT1 T cells. Both OT1-WT and OT1-KO T cells actively infiltrated tumors and showed an antigen-experienced phenotype (CD44^{hi}CD62L^{lo}; FIG. 2E, FIG. 10, C and D). However, OT1-KO T cells were more effective in controlling tumor growth, corresponding with their enhanced effector function and reduced exhaustion phenotype (FIG. 2F, FIG. 10, E to H). Collectively, these in vivo studies demonstrate that MAO-A works as an autonomous factor directly regulating CD8 T cell antitumor immunity.

### MAO-A Restrains the CD8 T Cell Response to Antigen Stimulation

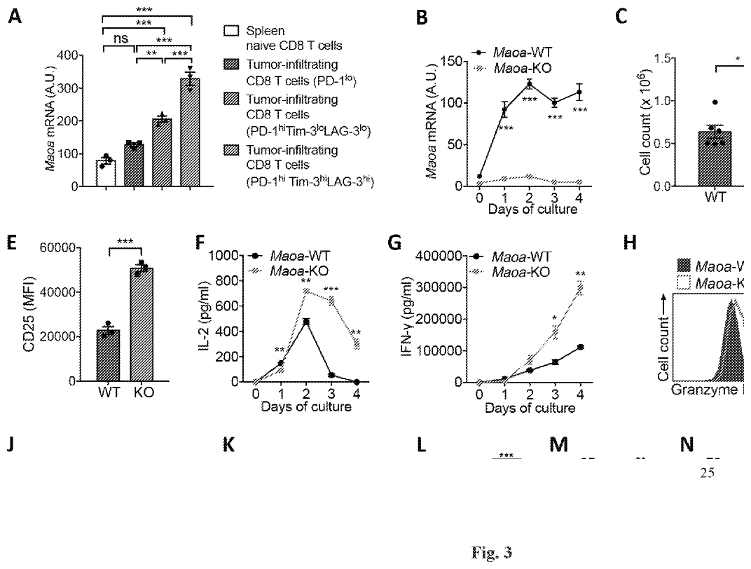

Analysis of Maoa mRNA expression in tumor-infiltrating CD8 T cells showed an induction of the Maoa gene in these T cells compared to naïve CD8 T cells (FIG. 3A). Further analysis of tumor-infiltrating CD8 T cell subsets revealed that Maoa gene expression levels were positively correlated with the exhaustion and dysfunction status of these T cells: compared to PD-1^{lo} cells, PD-1^{hi} “exhausted” T cells expressed higher levels of Maoa mRNA; and the PD-1^{hi} cells co-expressing TIM-3 and LAG-3, that were considered “most exhausted”, expressed the highest levels of Maoa mRNA (FIG. 3A) (16, 17). These in vivo data suggest that Maoa may be induced by tumor antigen recognition then act as a negative-feedback regulator inhibiting CD8 T cell antitumor reactivity.

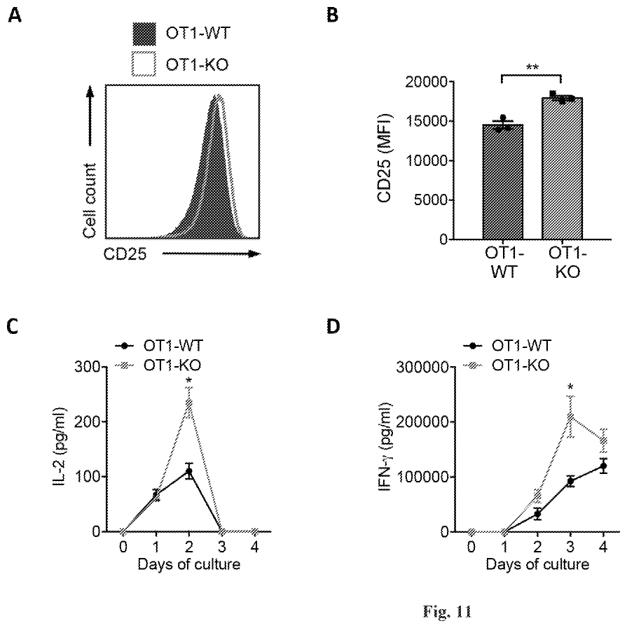

To test this hypothesis, we isolated CD8 T cells from Maoa-WT or Maoa-KO mice, and then stimulated these T cells in vitro with anti-CD3 mimicking tumor antigen stimulation. We observed an induction of Maoa mRNA expression in Maoa-WT CD8 T cells, in agreement with previous reports; minimal Maoa mRNA expression was detected in Maoa-KO CD8 T cells, confirming their Maoa-deficiency phenotype (FIG. 3B) (18). Compared to their Maoa-WT counterparts, Maoa-KO CD8 T cells showed an enhancement in almost all aspects of T cell activation, including cell proliferation (FIG. 3C), surface activation marker upregulation (i.e., CD25; FIG. 3, D and E), effector cytokine production (i.e., IL-2 and IFN-γ; FIG. 3, F and G), and cytotoxic molecule production (i.e., Granzyme B; FIG. 3, H and I). Study of OVA-specific OT1-KO T cells gave similar results (FIG. 11, A to D), suggesting a general role of MAO-A in regulating CD8 T cells of diverse antigen specificities. To verify whether MAO-A deficiency directly contributed to the hyperresponsiveness of the Maoa-KO CD8 T cells, we performed a rescue experiment. We constructed a MIG-Maoa retroviral vector, used this vector to transduce Maoa-KO CD8 T cells, and achieved overexpression of MAO-A in these T cells (FIG. 3, J to L). MAO-A overexpression significantly reduced the hyperactivation of Maoa-KO CD8 T cells and their expression of multiple effector genes (i.e., 112, Ifng, and Gzmb; FIG. 3, M to O). Taken together, these results indicate that MAO-A acts as a negative-feedback regulator restraining the CD8 T cell response to antigen stimulation.

### MAO-A Regulates CD8 T Cell Autocrine Serotonin Signaling

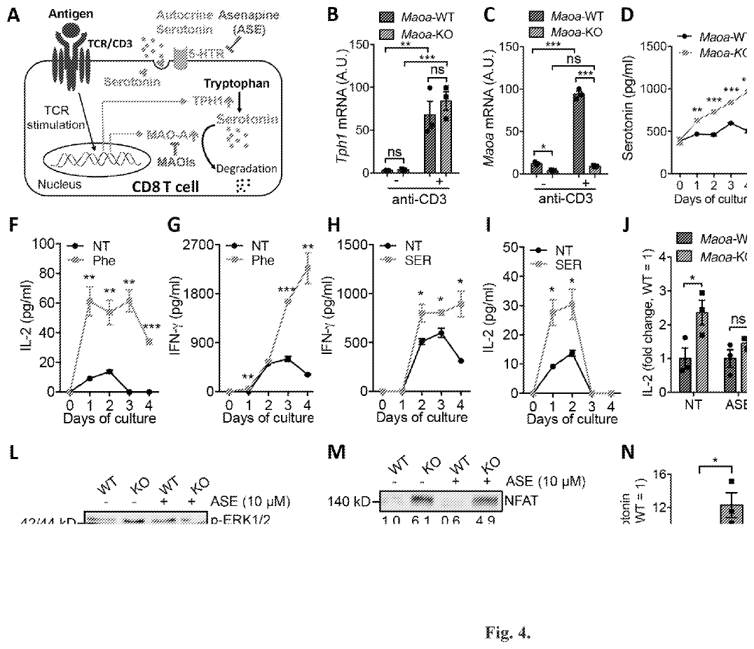

Next, we sought to investigate the molecular mechanisms mediating MAO-A restraint of CD8 T cell response to antigen stimulation. MAO-A is well known for its function in brain where it breaks down neuron-produced serotonin thereby regulating neuronal activity (8, 9). CD8 T cells have been reported to synthesis serotonin, and serotonin has been implicated as an accessory signal to enhance T cell activation by signaling through T cell surface serotonin receptors (5-HTRs) (18-20). We therefore postulated that MAO-A might regulate CD8 T cell activity through modulating T cell autocrine serotonin production and signaling (FIG. 4A).

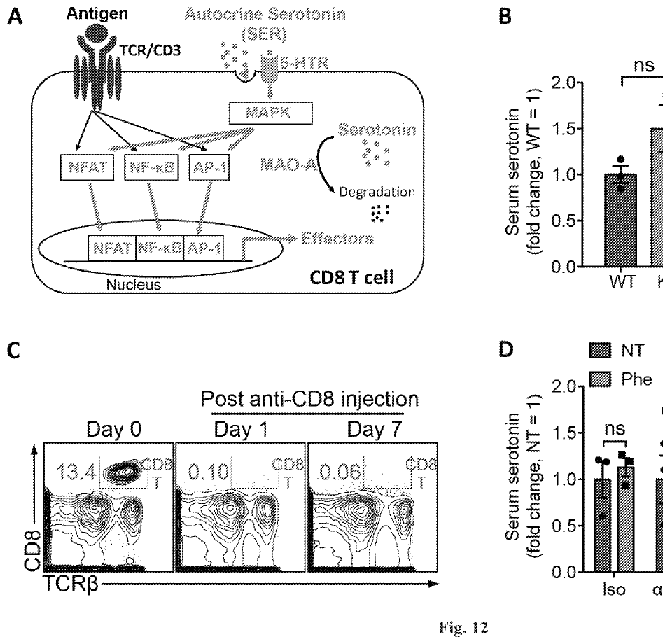

To test this hypothesis, we cultured Maoa-WT and Maoa-KO CD8 T cells in vitro, stimulated them with anti-CD3 to mimic antigen stimulation, and then analyzed their autocrine serotonin signaling pathway. Post antigen stimulation, Maoa-WT CD8 T cells upregulated expression of the Tph-1 gene, which encodes the rate-limiting enzyme controlling serotonin synthesis, and also upregulated expression of the Maoa gene, which would induce serotonin degradation, indicating the presence of an antigen stimulation-induced serotonin synthesis/degradation loop in CD8 T cells (FIG. 4, A to C). Considering the function of MAO-A, we speculated that MAO-A deficiency would not interfere with the serotonin synthesis arm but would impede the serotonin degradation arm, leading to enhanced secretion of serotonin by CD8 T cells. Indeed, compared to their wildtype counterparts, Maoa-KO CD8 T cells expressed comparable levels of Tph-1 but secreted much higher levels of serotonin post antigen stimulation (FIG. 4, B and D). Pharmacological inhibition of MAO-A in Maoa-WT CD8 T cells using an established MAO inhibitor (MAOI), phenelzine, recapitulated the serotonin overproduction phenotype of Maoa-KO CD8 T cells (FIG. 4E). Correspondingly, phenelzine treatment of Maoa-WT CD8 T cells recapitulated the hyperactivation phenotype of Maoa-KO CD8 T cells, shown by increased production of the effector cytokines IL-2 and IFN-γ (FIG. 4, F and G). Supplementing serotonin to Maoa-WT CD8 T cells resulted in T cell hyperactivation and elevated production of IL-2 and IFN-γ (FIG. 4, H and I), while blocking T cell surface serotonin receptors (5-HTRs) using an antagonist asenapine eliminated the cytokine production difference between Maoa-WT and Maoa-KO CD8 T cells (FIG. 4, J and K). Serotonin has been reported to enhance T cell activation by signaling through the MAPK pathway that cross-talks with the T cell receptor (TCR) signaling pathways (19). We compared the signaling pathways in Maoa-WT and Maoa-KO CD8 T cells post antigen stimulation and found that Maoa-KO T cells showed an enhancement of MAPK signaling (i.e., ERK phosphorylation; FIG. 4L) and TCR downstream signaling (i.e., nuclear translocation of NFAT, NF-κB, and c-Jun transcription factors; FIG. 4M); this enhancement was largely abrogated by blocking 5-HTRs (FIG. 4, L and M). Collectively, these in vitro data suggest that MAO-A regulates CD8 T cell activation through modulating T cell autocrine serotonin production and signaling (FIG. 4A, FIG. 12A).

To validate this working model in vivo, we directly measured intratumoral serotonin in Maoa-KO and Maoa-WT mice, as well as in wildtype mice treated or untreated with phenelzine. Consistent with the in vitro results, increased levels of serotonin were detected specifically in tumors collected from the Maoa-KO mice (FIG. 4N) and phenelzine-treated Maoa-WT mice (FIG. 4O). Depletion of CD8 T cells in Maoa-WT mice largely abolished the phenelzine treatment-induced accumulation of serotonin in the tumor, indicating that tumor-infiltrating CD8 T cells are major producers of serotonin in the tumor, and that this is negatively regulated by MAO-A (FIG. 4O, FIG. 12C). Notably, there were no significant changes in serotonin levels in serum under any conditions (FIG. 12, B and D), suggesting that MAO-A regulation of serotonin in the tumor is largely a local effect, resembling MAO-A regulation of serotonin in the brain (8, 9).

Taken together, these in vitro and in vivo data support a working model that MAO-A negatively regulates CD8 T cell antitumor immunity, at least partly through modulating CD8 T cell autocrine serotonin signaling in the tumor.

### MAO-A Blockade for Cancer Immunotherapy

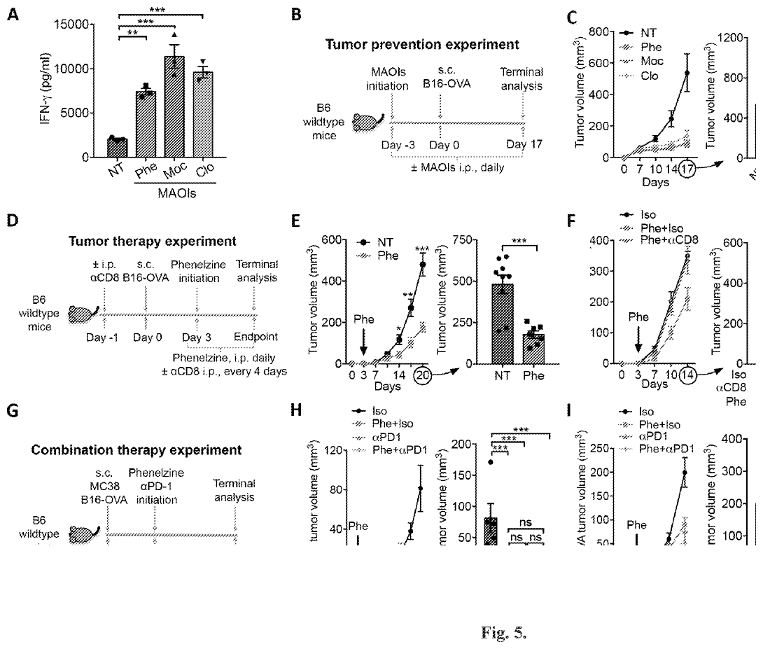

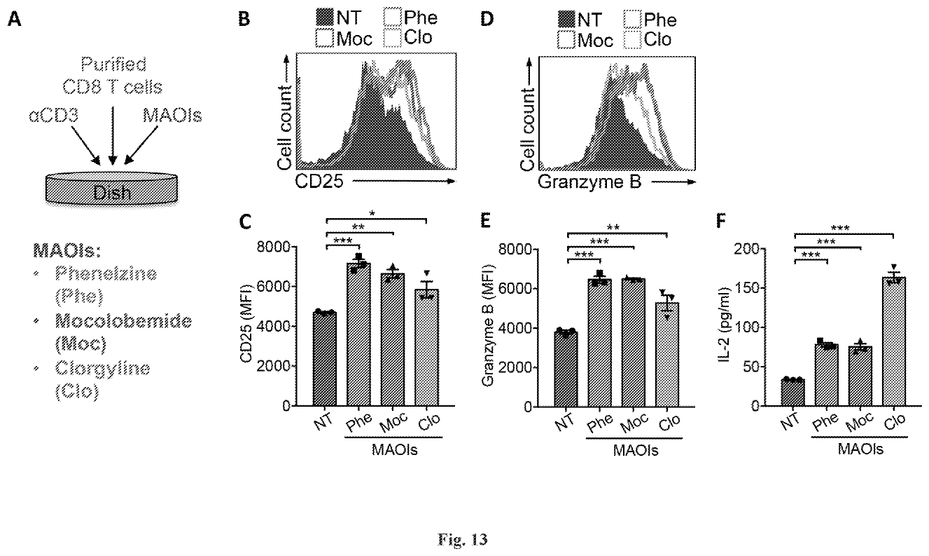

The identification of MAO-A as a new immune checkpoint negatively regulating CD8 T cell antitumor immunity marks it as a promising drug target for developing new forms of ICB therapy. Because of MAO-A's well-characterized function in the brain, small molecule MAOIs have been developed and clinically utilized for treating depression symptoms, making it a highly feasible and attractive approach to repurpose these established MAOI antidepressants for cancer immunotherapy (21). Notably, some MAOIs cross-inhibit the MAO-A isoenzyme MAO-B; however, only MAO-A effectively degrades serotonin, and all MAOIs exhibit their antidepressant function mainly through inhibiting MAO-A enzyme activity thereby regulating serotonin signaling in the brain (14, 21). When tested in vitro, multiple MAOIs efficiently induced CD8 T cell hyperactivation (i.e., upregulated expression of CD25, Granzyme B, IL-2, and IFN-γ; FIG. 5A and FIG. 13, A to F). When tested in vivo in a B16-OVA melanoma prevention model, these MAOIs dramatically suppressed tumor growth (FIG. 5, B and C). Notably, the MAOIs that we tested were phenelzine, clorgyline, and mocolobemide, covering the major categories of established MAOIs classified on the basis of whether they are nonselective or selective for MAO-A, and whether their effect is reversible (FIG. 13A) (21). Among these MAOIs, phenelzine (trade name: Nardil) is clinically available in the United States (21). In the following studies, we chose phenelzine as a representative to further evaluate the cancer therapy potential of MAOI drugs.

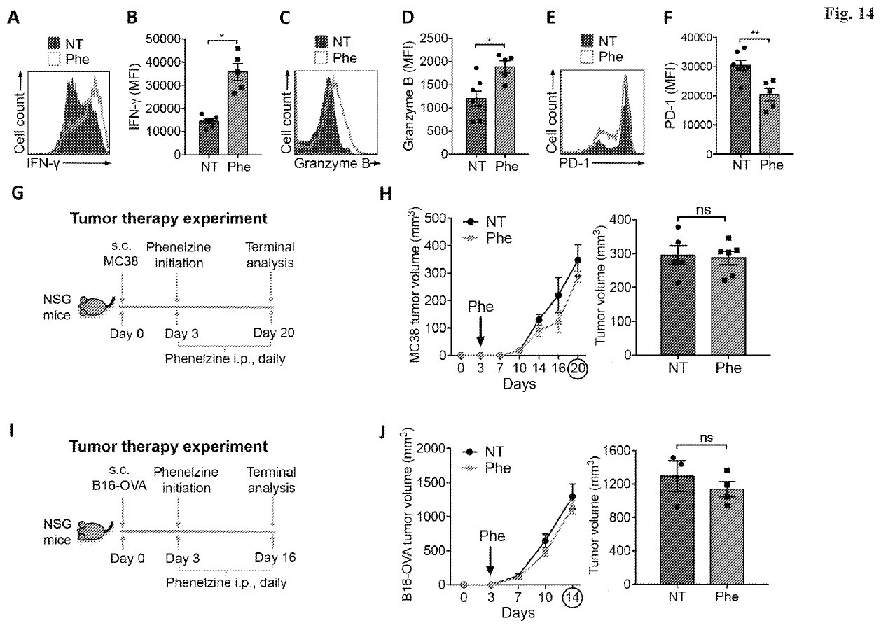

First, we studied the efficacy of phenelzine in treating pre-established B16-OVA melanoma solid tumors, and found that phenelzine treatment effectively suppressed tumor progression (FIG. 5, D and E). This MAOI-induced tumor suppression effect was mediated by CD8 T cells, because no tumor suppression was observed when we depleted CD8 T cells in tumor-bearing B6 wildtype mice (FIG. 5F). Correspondingly, analysis of tumor-infiltrating CD8 T cells under phenelzine treatment showed a hyperactivation phenotype of these T cells, evidenced by their enhanced production of effector molecules (i.e., IFN-γ and Granzyme B; FIG. 14, A to D) and reduced expression of exhaustion markers (i.e., PD-1; FIG. 14, E and F). Next, we studied the potential of phenelzine treatment for combination therapy, in particular combining with other ICB therapies such as the PD-1/PD-L1 blockade therapy (FIG. 5G) (6). In the MC38 colon cancer model, which is sensitive to immunotherapy, phenelzine treatment completely suppressed tumor growth as effectively as the anti-PD-1 treatment (FIG. 5H). In the B16-OVA melanoma model, which is less sensitive to immunotherapy, phenelzine treatment significantly suppressed tumor growth at a level comparable to the anti-PD-1 treatment; importantly, the combination of phenelzine and anti-PD-1 treatments yielded supreme efficacy and totally suppressed tumor growth (FIG. 5I). These tumor suppression effects of phenelzine were mediated by its immune regulatory function, because phenelzine treatment did not suppress the growth of MC38 and B16-OVA tumors in immunodeficient NSG mice (FIG. 14, G to J). Collectively, these syngeneic mouse tumor model studies provided proof-of-principle evidence for the cancer immunotherapy potential of MAOIs.

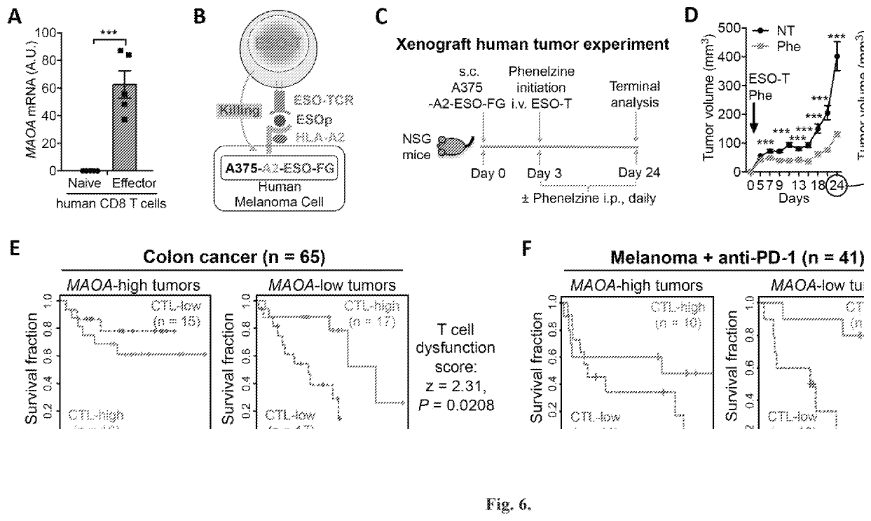

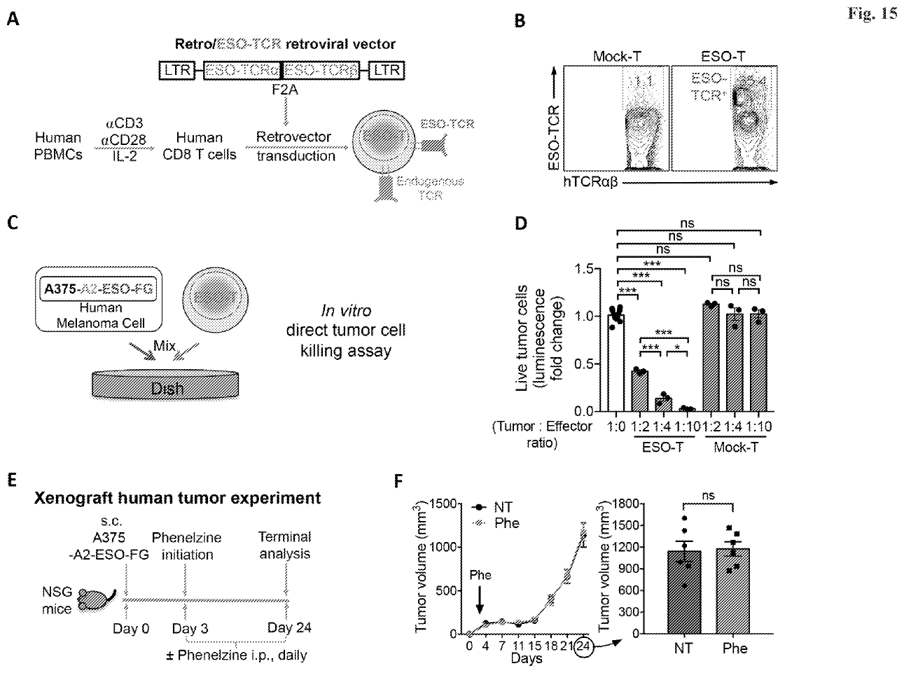

To explore the translational potential of MAO-A blockade therapy, we studied human CD8 T cells and confirmed that they also upregulated MAOA gene expression post antigen stimulation, resembling their mouse counterparts (FIG. 6A). To directly evaluate whether MAOI treatment could enhance human CD8 T cell antitumor efficacy in vivo, we utilized a human T cell adoptive transfer and a human tumor xenograft NSG mouse model (22). NY-ESO-1, a well-recognized tumor antigen common in many human cancers, was chosen as the model tumor antigen (22). An A375 human melanoma cell line was engineered to co-express NY-ESO-1 as well as its matching MHC molecule, HLA-A2, to serve as the human tumor target; the cell line was also engineered to express a dual reporter comprising a firefly luciferase and an enhanced green fluorescence protein (denoted as A375-A2-ESO-FG) (23). A Retro/ESO-TCR retroviral vector was constructed to encode an NY-ESO-1 specific TCR (clone 3A1; denoted as ESO-TCR) and was used to transduce healthy donor peripheral blood CD8 T cells; the resulting T cells (denoted as ESO-T cells) expressed ESO-TCRs and specifically targeted A375-A2-ESO-FG tumor cells, thereby modeling the tumor-specific human CD8 T cells (FIG. 6B and FIG. 15, A to D). A375-A2-ESO-FG cells were subcutaneously injected into NSG mice to establish solid tumors, followed by intravenous injection of ESO-T cells with or without phenelzine treatment (FIG. 6C). MAOI treatment effectively suppressed tumor growth; this therapeutic effect was mediated by tumor-specific ESO-T cells because no tumor suppression was observed in NSG mice that did not receive adoptive transfer of ESO-T cells (FIG. 6D, FIG. 15, E and F). Collectively, this human xenograft tumor model study supports the translational potential of MAO-A blockade for cancer immunotherapy.

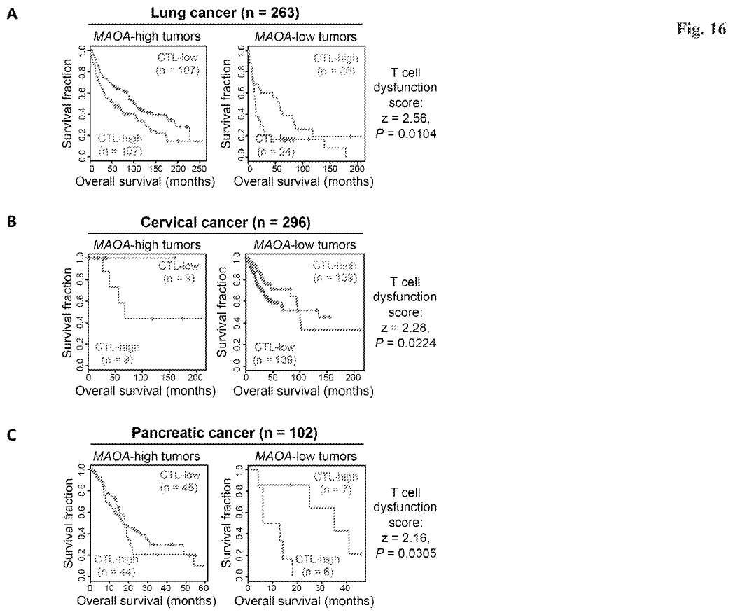

Lastly, we conducted clinical data correlation studies to investigate whether MAOA gene expression is correlated with CD8 T cell (cytotoxic T lymphocyte, CTL) antitumor activities and clinical outcomes in cancer patients. A Tumor Immune Dysfunction and Exclusion (TIDE) computational method was used, that models the induction of CD8 T cell dysfunction in tumors by analyzing the interactions of three variants: 1) the intratumoral expression of a selected gene, 2) the intratumoral level of CD8 T cells, and 3) patient survival (24). MAOA expression level was negatively correlated with the beneficial effect of tumor-infiltrating CD8 T cell on patient survival in multiple cancer patient cohorts spanning colon cancer (FIG. 6E) (25), lung cancer (FIG. 16A) (26), cervical cancer (FIG. 16B) (27), and pancreatic cancer (FIG. 16C) (28). Moreover, analysis of a melanoma patient cohort receiving anti-PD-1 treatment showed that high levels of intratumoral MAOA expression largely abrogated the patient survival benefit offered by tumor-infiltrating CD8 T cells, suggesting that combining MAO-A blockade therapy with PD-1/PD-L1 blockade therapy may provide synergistic therapeutic benefits through further activating tumor-infiltrating CD8 T cells (FIG. 6F) (29). Of note, these whole tumor lysate transcriptome data analyses could not localize the MAOA expression to a specific cell type (i.e., CD8 T cells); future studies of quality transcriptome data generated from single cells or sorted tumor-infiltrating CD8 T cells are needed to produce such information. Nonetheless, the present clinical data correlation studies identified MAO-A as a possible negative regulator of CD8 T cell antitumor function in a broad range of cancer patients, including those receiving existing ICB therapies, suggesting MAO-A as a potential drug target for developing new forms of ICB therapy and combination therapy.

Taken together, these preclinical animal studies and clinical data correlation studies suggest that MAO-A is a promising new drug target of T cell-based cancer immunotherapy, and that repurposing of established MAOI antidepressants is a promising path to develop MAO-A blockade immunotherapy.

### Discussion

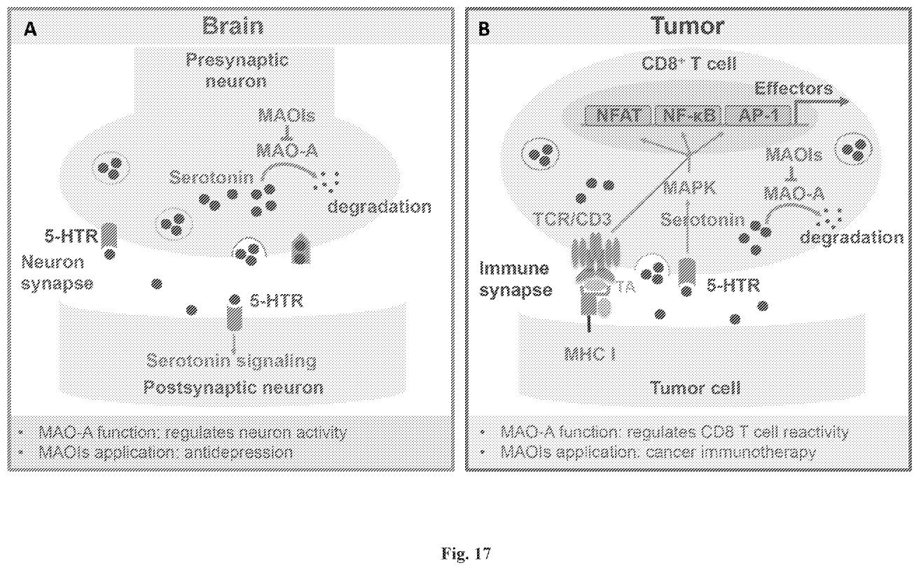

Based on our findings, we propose an “intratumoral MAO-A-serotonin axis” model to elucidate the role of MAO-A in regulating CD8 T cell antitumor immunity (FIG. 17). Analogous to the well-characterized MAO-A-serotonin axis in the brain, where MAO-A controls the availability of serotonin in a neuron-neuron synapse thereby regulates neuronal activity, the “MAO-A-serotonin axis” in tumor controls the availability of serotonin in a tumor-T cell immune synapse thereby regulates antitumor T cell reactivity (FIG. 17). The resemblance is notable, considering that both the nervous system and the immune system are evolved to defend a living organism by sensing and reacting to environmental danger, externally and internally, including tissue traumas, infections, and malignancies (30). Despite their distinct anatomic structures—the nervous system has a fixed organization while the immune system comprises mobile and disperse cells—from an evolutionary point of view, it makes sense that some critical molecular regulatory pathways are preserved for both defense systems. Indeed, neurons and immune cells share a broad collection of signal transducers, surface receptors, and secretory molecules (30). In particular, many neurotransmitters and neuropeptides traditionally considered specific for neurons are expressed in immune cells, although their functions in the immune system are to a large extent still unknown (31). Studying this group of molecules and their regulatory circuits in tumor immunology thus may provide new opportunities for generating knowledge and identifying new drug targets for developing next-generation cancer immunotherapies; our current finding of this “MAO-A-serotonin axis” regulation of CD8 T cell antitumor immunity can be such an example.

Our study showed that Maoa expression was induced by antigen-TCR stimulation in CD8 T cells and in turn restrained T cell activation (FIG. 3). This negative-feedback loop qualifies MAO-A as an immune checkpoint and adds it to the expanding immune checkpoint family comprising PD-1/PD-L1, CTLA-4, TIM-3, LAG-3, TIGIT, VISTA, and others (4). However, MAO-A is unique in this group because it is already a well-established drug target due to its known function in the brain (21). In fact, small molecule MAOIs have been developed to block MAO-A activity thereby regulating serotonin signaling in the brain and were the first drugs approved for treating depression (21). In our study, we tested multiple clinically approved MAOIs (phenelzine, moclobemide, and clorgyline) and demonstrated their T cell-enhancing and tumor suppression effects in preclinical animal models, pointing to the possibility of repurposing these drugs for cancer immunotherapy (FIG. 5, and FIG. 13). Developing new cancer drugs is extremely costly and time-consuming; drug repurposing offers an economic and speedy pathway to novel cancer therapies, because approved drugs have known safety profiles and modes of actions, thus can enter the clinic quickly (32). MAOIs were introduced in the 1950s and were used extensively over the subsequent two decades, but since then their use has dwindled because of reported side effects and the introduction of other classes of antidepressant agents (21). However, these MAOI side effects were vastly overstated and should be revisited (21). For instance, a claimed major side effect of MAOIs is their risk of triggering tyramine-induced hypertensive crisis when patients eat tyramine-rich foods such as aged cheese (hence, “the cheese effects”); this concern led to cumbersome food restrictions that is now considered largely unnecessary (21). A transdermal delivery system (Emsam) has also been developed to deliver MAOIs that can largely avoid potential food restrictions (21). Therefore, interest in MAOIs as a major class of antidepressants is reviving (21), and repurposing MAOIs for cancer immunotherapy can be an attractive new application of these potent drugs.

Depression and anxiety are common in cancer patients: prevalent rates of major depression among cancer patients are four times higher than the general population, and up to a quarter of cancer patients have clinically significant depression and anxiety symptoms (33). Repurposing MAOIs for cancer immunotherapy thus may provide cancer patients with dual antidepression and antitumor benefits. Notably, a large majority of antidepressants, including MAOIs, SSRIs (selective serotonin reuptake inhibitors), SMSs (serotonin modulators and stimulators), SARIs (serotonin antagonists and uptake inhibitors), and SNRIs (serotonin-norepinephrine reuptake inhibitors), all work through regulating serotonin signaling in the brain via inhibiting the various key molecules that control serotonin degradation, reuptake, and detection (34). Our study revealed the existence of a “MAO-A-serotonin axis” in tumors that regulates CD8 T cell antitumor immunity (FIG. 17). It is plausible to postulate that the other key serotonin regulatory molecules that function in the brain may also function in the tumor regulating T cell antitumor immunity (34). Intriguingly, a recent nationwide cohort study in Israel reported that adherence to antidepressant medications is associated with reduced premature mortality in patients with cancer (33). Another clinical study reported lymphocyte subset changes associated with antidepressant treatments in patients with a major depression disorder (35). Studying cancer patients for possible correlations between antidepressant treatments, antitumor immune responses, and clinical outcomes therefore may yield valuable knowledge informing the immune regulatory function of antidepressants and instructing the repurposing of a large collection of antidepressant drugs for cancer immunotherapy.

In our study, we found that Maoa gene highly expressed in tumor-infiltrating CD8 T cells, with the most “exhausted” cells (PD-1^{hi}Tim-3^{hi}LAG-3^{hi}) expressing the highest levels of Maoa, suggesting that these cells may benefit the most from the MAOI treatment (16, 17). We also found that MAO-A regulated CD8 T cell antitumor immunity at least partly through modulating the serotonin-MAPK pathway, which is non-redundant to other major immune checkpoint regulatory pathways, suggesting that MAOI treatment can be a valuable component for combination therapy (4). Indeed, MAOI treatment synergized with anti-PD-1 treatment in suppressing syngeneic mouse tumor growth, and MAOA expression levels dictated patient survival in melanoma patients receiving anti-PD-1 therapy (FIGS. 5 and 6). Notably, patients undergoing cancer treatment, including traditional chemo/radio therapies as well as the new immunotherapies like ICB therapies, often report incurred or exacerbated depression symptoms; these CNS (central nervous system) side effects are considered to be associated with treatment-induced immune reaction and inflammation (36-38). Adding MAOIs with antidepression function to a combination cancer therapy thus may both improve antitumor efficacy and alleviate CNS side effects. Interestingly, some earlier studies reported that MAOIs can directly suppress the growth of androgen-sensitive and castration-resistant prostate cancer cells, presumably through regulating cancer cell autophagy and apoptosis, suggesting additional mechanisms that MAOIs may deploy to target certain cancers (39, 40).

In summary, here we identified MAO-A as an immune checkpoint, and demonstrated the potential of repurposing established MAOI antidepressants for cancer immunotherapy. The notion that MAOA the “warrior gene” not only takes action in the brain regulating the aggressiveness of human behavior, but also takes action in the tumor regulating the aggressiveness of antitumor immunity, is interesting. Future clinical studies are encouraged to investigate the clinical correlations between MAOI treatment and clinical outcomes in cancer patients, and to explore the possibility of repurposing MAOIs for combination cancer immunotherapy. Meanwhile, the immune regulatory function of MAO-A certainly goes beyond regulating CD8 T cells. In Maoa-KO mice, we have observed the hyperresponsiveness of multiple immune cells in various mouse tumor models. It is also likely that MAO-A regulates immune reactions to multiple diseases beyond cancer, such as infections and autoimmune diseases. Studying the roles of MAO-A in regulating various immune cells under different health and disease conditions will be interesting topics for future research.

### Materials and Methods

### Mice

C57BL/6J (B6), B6.SJL-Ptprc^{a}Pepc^{b}/BoyJ (CD45.1, BoyJ), 129S-Maoa^{tm1Shih}/J (Maoa-KO) (15), C57BL/6-Tg (TcraTcrb)1100Mjb/J (OT1-Tg), and NOD.Cg-Prkdc^{scid} Il2rg^{tm1Wj1}/SzJ (NSG) mice were purchased from the Jackson Laboratory (Bar Harbor). The OT1-Tg mice deficient of Maoa (OT1-Tg/Maoa-KO) were generated at the University of California, Los Angeles (UCLA) through breeding OT1-Tg mice with Maoa-KO mice. All animals were maintained in the animal facilities at UCLA. Eight- to twelve-week-old females were used for all experiments unless otherwise indicated. All animal experiments were approved by the Institutional Animal Care and Use Committee of UCLA.

### Cell Lines

The B16-OVA mouse melanoma cell line and the PG13 retroviral packaging cell line were provided by Dr. Pin Wang (University of South California, CA) (41). The MC38 mouse colon adenocarcinoma cell line was provided by Dr. Antoni Ribas (UCLA) (42). The HEK 293T and Phoenix-ECO retroviral packaging cell lines were purchased from the American Type Culture Collection (ATCC). The A375-A2-ESO-FG human melanoma cell line was previously reported (22, 23). The Phoenix-ECO-MIG, Phoenix-ECO-MIG-Maoa, and PG13-ESO-TCR stable virus producing cell lines were generated in this study.

### Viral Vectors

The MIG (MSCV-IRES-GFP) retroviral vector was reported previously (43). MIG-Maoa and Retro/ESO-TCR retroviral vectors were generated in this study.

### Media and Reagents

Adherent cell culture medium (denoted as D10 medium) was made of Dulbecco's modified Eagle's medium (DMEM, Cat #10013, Corning) supplemented with 10% fetal bovine serum (FBS, Sigma) and 1% Penicillin-Streptomycin-Glutamine (Cat #10378016, Gibco). T cell culture medium (denoted as C10 medium) was made of RPMI 1640 (Cat #10040, Corning) supplemented with 10% FBS (Cat #F2442, Sigma), 1% Penicillin-Streptomycin-Glutamine (Cat #10378016, Gibco), 0.2% Normocin (Cat #ant-nr-2, Invivogen), 1% MEM Non-Essential Amino Acids Solution (Cat #11140050, Gibco), 1% HEPES (Cat #15630080, Gibco), 1% Sodium Pyruvate (Cat #11360070, Gibco), and 0.05 mM β-Mercaptoethanol (Cat #M3148, Sigma).

Cell culture reagents, including purified NA/LE anti-mouse CD3ε (Cat #553057, clone 145-2C11), anti-human CD3 (Cat #56685, clone OKT3), and anti-human CD28 (Cat #555725, clone CD28.2), were purchased form BD Bioscience. Recombinant human IL-2 (Cat #200-02) was purchased from PeproTech.

In vivo depletion antibodies, including anti-mouse CD8α (Cat #BE0061, clone RMP2.43) and its isotype control (rat IgG2b, cat #BE0090), were purchased from BioXCell. In vivo PD-1 blocking antibody (Cat #BE0146, clone RMP1-14) and its isotype control (rat IgG2a, cat #BE0089) were purchased from BioXCell.

Monoamine oxidase inhibitors (MAOIs), including phenelzine (Cat #P6777), moclobimide (Cat #M3071), and clorgyline (Cat #M3778), were purchased from Sigma. Serotonin (Cat #H9532) and serotonin receptor (5-HTR) antagonist asenapine (Cat #A7861) were also purchased from Sigma.

### Syngeneic Mouse Tumor Models

B16-OVA melanoma cells (1×10⁶ per animal) or MC38 colon cancer cells (3×10⁵ per animal) were subcutaneously (s.c.) injected into experimental mice to form solid tumors. In some experiments, mice received intraperitoneal (i.p.) injection of MAOIs (i.e., phenelzine, 30 mg/kg/day; moclobimide, 50 mg/kg/day; or clorgyline, 50 mg/kg/day) to block MAO-A activity. In some experiments, mice received i.p. injection of anti-mouse CD8α antibodies (200 μg/animal/bi-weekly) to deplete CD8 T cells; mice received i.p. injection of isotype antibodies were included as controls. In some experiments, mice received i.p. injection of anti-mouse PD-1 antibodies (300 μg/animal/bi-weekly) to block PD-1; mice received i.p. injection of isotype antibodies were included as controls.

During an experiment, tumor growth was monitored twice per week by measuring tumor size using a Fisherbrand™ Traceable™ digital caliper (Thermo Fisher Scientific); tumor volumes were calculated by formula ½×L×W². At the end of an experiment, tumor-infiltrating immune cells were isolated for analysis using QPCR, flow cytometry, and/or scRNASeq. In some experiments, sera were also collected for serotonin measurement.

### Two-Way Bone Marrow (BM) Transfer B16-OVA Tumor Model

BM cells were collected from femurs and tibias of donor mice, and were transfer into the recipient mice through retrol orbital (r.o.) injection. Recipient mice were preconditioned with whole body irradiation (1200 rads). For BM transfer experiments confining MAO-A deficiency comparison in immune cells, Maoa-WT or Maoa-KO BM cells were transferred into BoyJ recipient mice (8-10×10⁶ cells per recipient mouse). For BM transfer experiments confining MAO-A deficiency comparison in non-immune cells, WT BoyJ bone marrow cells were transferred into Maoa-WT or Maoa-KO recipient mice (8-10×10⁶ cells per recipient mouse). After BM transfer, recipient mice were maintained on antibiotic water (Amoxil, 0.25 mg/ml) for 4 weeks. Periodic bleedings were performed to monitor immune cell reconstitution using flow cytometry. At 8 to 12 weeks post BM transfer, recipient mice were fully immune reconstituted, and were used for tumor challenge experiments. B16-OVA mouse melanoma cells were s.c. injected into experimental mice to form solid tumors (1×10⁶ cells per animal). Tumor growth was monitored twice per week by measuring tumor size using a Fisherbrand™ ‘ Traceable’ digital caliper; tumor volumes were calculated by formula ½×L×W².

### Adoptive OT1 T Cell Transfer B16-OVA Tumor Model

Spleen and lymph node cells were harvested from the OT1-Tg or OT1-Tg/Maoa-KO mice, and were subjected to magnetic-activated cell sorting (MACS) using a Mouse CD8 T Cell Isolation Kit (Cat #120117044, Miltenyi Biotec) following the manufacturer's instructions. The purified OT1 T cells (identified as CD8⁺TCR Vβ5⁺ cells) were adoptively transferred to tumor-bearing BoyJ wildtype mice (1×10⁵ cells per recipient mouse). BoyJ mice were s.c. inoculated with B16-OVA tumor cells one week in advance (1×10⁶ cells per animal). Prior to OT1 T cell adoptive transfer, recipient mice were preconditioned with whole body irradiation (600 rads). During an experiment, tumor growth was monitored twice per week by measuring tumor size using a Fisherbrand™ Traceable™ digital caliper; tumor volumes were calculated by formula ½×L×W². Mice were terminated at the indicated time points, and TIIs were isolated for flow cytometry analysis of surface marker expression and intracellular effector molecule production.

### Xenograft Human Tumor Model

The A375-A2-ESO-FG human melanoma cells (10×10⁶ cells per animal) were s.c. injected into NSG mice to form solid tumors. In some experiments, mice received phenelzine treatment through i.p. injection (30 mg/kg/day). In some experiments, mice received adoptive transfer of ESO-T cells through r.o. injection (4×10⁶ cells per recipient mouse). Prior to ESO-T cell adoptive transfer, recipient mice were preconditioned with total body irradiation (100 rads). During an experiment, tumor growth was monitored twice per week by measuring tumor size using a Fisherbrand™ Traceable™ digital caliper; tumor volumes were calculated by formula ½×L×W².

### Tumor-Infiltrating Immune Cell (TH) Isolation and Analysis

Solid tumors were harvested from experimental mice and mechanically disrupted through 70 μm nylon mesh strainers to release single cells (Cat #07-201-431, Corning). Single cells were washed once with C10 medium, resuspended in 50% percoll (Cat #P4937, Sigma), and centrifuged at 800 g at 25° C. for 30 min with brake off. Cell pellets enriched with TIIs were then collected and resuspended in C10 medium for further analysis.

In the experiment studying the Maoa gene expression in TIIs, day-14 B16-OVA tumors were harvested from B6 wildtype mice to prepare TII suspensions. TII suspensions were then sorted using a FACSAria II flow cytometer (BD Biosciences) to purify immune cells (gated as DAPI⁻CD45.2⁺ cells), which were then subjected to QPCR analysis of Maoa mRNA expression.

In the experiment studying the Maoa gene expression in tumor-infiltrating CD8 T cell subsets, day-14 B16-OVA tumors were harvested from B6 wildtype mice to prepare TII suspensions. Tumor-infiltrating CD8 T cells (pre-gated as CD45.2⁺TCRβ⁺CD8⁺ cells) were sorted into three subsets (gated as PD-1^{lo}, PD-1^{hi}LAG-3^{lo}Tim-3^{lo}, and PD-1^{hi}LAG-3^{hi}Tim-3^{hi} cells) using a FACSAria II flow cytometer, and then were subjected to QPCR analysis of Maoa mRNA expression.

In the experiment studying gene expression profiling of TIIs, day-14 B16-OVA tumors were harvested from Maoa-WT and Maoa-KO mice to prepare TII suspensions. TII suspensions were then sorted using a FACSAria II flow cytometer to purify immune cells (gated as DAPI⁻CD45.2⁺ cells), which were then subjected to scRNASeq analysis.

In other experiments, TII suspensions prepared under indicated experimental conditions were directly analyzed by flow cytometry to study surface marker expression and intracellular effector molecule production of CD8 T cells (pregated as CD45.2⁺TCRβ⁺CD8⁺ cells).

### In Vitro Mouse CD8 T Cell Culture

Spleen and lymph node cells were harvested from Maoa-KO or Maoa-WT (B6 wildtype) mice and subjected to MACS using a Mouse CD8 T Cell Isolation Kit (Cat #120117044, Miltenyi Biotec) following the manufacturer's instructions. Purified mouse CD8 T cells were cultured in vitro in C10 medium, in a 24-well plate at 0.5×10⁶ cells per ml medium per well, in the presence of plate-bound anti-mouse CD3ε (5 μg/ml) for up to 4 days. At indicated time points, cells were collected for flow cytometry analysis of surface marker expression and intracellular effector molecule production, and for QPCR analysis of mRNA expression; cell culture supernatants were collected for ELISA analysis of effector cytokine production.

In experiments studying serotonin signaling, cells were cultured in C10 medium made of serotonin-depleted FBS that was pretreated overnight with charcoal-dextran (Cat #C6241, Sigma; 1 gram per 50 ml FBS). L-Ascorbic acid (Cat #A4403, Sigma; 100 μM) was added to C10 medium to stabilize T cell-produced or supplemented serotonin. In some experiments, cells were treated with MAOIs to block MAO-A activity; MAOIs studied were phenelzine (Phe, 10 μM), moclobimide (Moc, 200 μM), or clorgyline (Clo, 20 μM). In some experiments, cells were supplemented with exogenous serotonin (SER, 10 μM) to stimulate serotonin signaling. In some experiments, cells were treated with serotonin receptor antagonist asenapine (ASE, 10 μM) to block serotonin receptor signaling.

### In Vitro Human CD8 T Cell Culture

Healthy donor human peripheral blood mononuclear cells (PBMCs) were purchased from the UCLA/CFAR Virology Core Laboratory. PBMCs were cultured in C10 medium in the presence of plate-bond anti-human CD3 (1 μg/ml) and soluble anti-human CD28 (1 μg/ml). After 5 days, activated CD8 T cells were sorted out based on surface markers (CD45⁺TCRαβ⁺CD8⁺) using a FACSAria II flow cytometer (BD Biosciences). Naïve CD8 T cells were sorted from the same donors based on surface markers (CD45⁺TCRαβ⁺CD8⁺CD62L^{hi}CD45RO^{low}) and were included as controls. The purified naïve and effector human CD8 T cells were then analyzed for MAOA mRNA expression using QPCR.

### In Vitro OT1 T Cell Culture

Spleen and lymph node cells were harvested from the OT1-Tg or OT1-Tg/Maoa-KO mice, and then subjected to MACS sorting using a Mouse CD8 T Cell Isolation Kit (Cat #120117044, Miltenyi Biotec) following the manufacturer's instructions. The purified OT1 T cells (identified as CD8⁺TCR Vβ5⁺ cells) were cultured in C10 medium, in a 24-well plate at 0.5×10⁶ cells per ml medium per well, in the presence of plate-bound anti-mouse CD3ε (5 μg/ml) for up to 4 days. At the indicated time points, cells were collected for flow cytometry analysis of surface marker expression; cell culture supernatants were collected for ELISA analysis of effector cytokine production.

### MIG-Maoa Retroviral Vector and Mouse CD8 T Cell Transduction

The MIG-Maoa retroviral vector was constructed by inserting a codon-optimized Maoa cDNA (synthesized by IDT) into the parental MIG retroviral vector (43). The Vsv-g-pseudotyped MIG and MIG-Maoa retroviruses were produced using HEK 293T virus packaging cells following a standard calcium precipitation method (44, 45), and then were used to transduce Phoenix-ECO cells to generate stable cell lines producing ECO-pseudotyped MIG or MIG-Maoa retroviruses (denoted as Phoenix-ECO-MIG and Phoenix-ECO-MIG-Maoa cell lines, respectively). For virus production, Phoenix-ECO-MIG and Phoenix-ECO-MIG-Maoa cells were seeded at a density of 0.8×10⁶ cells per ml in D10 medium, and cultured in a 15 cm-dish (30 ml per dish) for 2 days; virus supernatants were then harvested and freshly used for spin-infection.

MACS-purified CD8 T cells isolated from the Maoa-KO mice were cultured in vitro and stimulated with plate-bound anti-mouse CD3ε (5 μg/ml) for 4 days. On day 2 and day 3, cells were spin-infected with ECO-pseudotyped MIG or MIG-Maoa retroviral supernatants supplemented with polybrene (cat #TR-1003-G, Millipore; 10 μg/ml) at 1321 g at 30° C. for 90 min. On day 4, cells were collected for flow cytometry analysis of transduction efficiency and for QPCR analysis of effector gene expression.

### Retro/ESO-TCR Retroviral Vector and Human CD8 T Cell Transduction

The Retro/ESO-TCR vector was constructed by inserting into the parental pMSGV vector a synthetic gene encoding an HLA-A2-restricted, NY-ESO-1 tumor antigen-specific human CD8 TCR (clone 3A1) (22). Vsv-g-pseudotyped Retro/ESO-TCR retroviruses were generated by transfecting HEK 293T cells following a standard calcium precipitation protocol and an ultracentrifugation concentration protocol (46); the viruses were then used to transduce PG13 cells to generate a stable retroviral packaging cell line producing GALV-pseudotyped Retro/ESO-TCR retroviruses (denoted as PG13-ESO-TCR cell line). For virus production, the PG13-ESO-TCR cells were seeded at a density of 0.8×10⁶ cells per ml in D10 medium, and cultured in a 15 cm-dish (30 ml per dish) for 2 days; virus supernatants were then harvested and stored at −80° C. for future use.

Healthy donor PBMCs were stimulated with plate-bound anti-human CD3 (1 μg/mL) and soluble anti-human CD28 (1 μg/mL) in the presence of recombinant human IL-2 (300 U/mL). On day 2, cells were spin-infected with frozen-thawed Retro/ESO-TCR retroviral supernatants supplemented with polybrene (10 μg/ml) at 660 g at 30° C. for 90 min following an established protocol (23). Transduced human CD8 T cells (denoted as ESO-T cells) were expanded for another 7-10 days, and then cryopreserved for future use. Mock-transduced human CD8 T cells (denoted as Mock-T cells) were generated as controls.

### In Vitro A375-A2-ESO-FG Human Melanoma Cell Killing Assay

The A375-A2-ESO-FG human melanoma cells (5-10×10³ cells per well) were co-cultured with either ESO-T cells or Mock-T cells at indicated ratios in C10 medium in a Corning 96-well clear bottom black plate (Cat #3603, Corning). At 24-hour, live tumor cells were quantified by adding D-Luciferin (Part #119222, Caliper Life Science; 150 μg/ml) to cell cultures and reading out luciferase activities using an Infinite M1000 microplate reader (Tecan) according to the manufacturer's instructions.

### Flow Cytometry

Flow cytometry, also known as FACS (fluorescence-activated cell sorting), was used to analyze surface marker and intracellular effector molecule expression of T cells. Fluorochrome-conjugated monoclonal antibodies specific for mouse CD45.2 (clone 104), TCRβ (clone H57-597), CD4 (clone RM4-5), CD8 (clone 53-6.7), CD69 (clone H1.2F3), CD25 (clone PC61) CD44 (clone IM7), CD62L (clone MEL-14), IFN-γ (clone XMG1.2), were purchased from BioLegend. Monoclonal antibodies specific for mouse TNF-α (clone JES6-5H4) and Fc block (anti-mouse CD16/32) (clone 2.4G2) were purchased from BD Biosciences. Monoclonal antibodies specific for mouse PD-1 (clone RMP1-30) was purchased from Thermo Fisher Scientific. Fluorochrome-conjugated monoclonal antibodies specific for human CD45 (clone H130), TCRαβ (clone I26), CD4 (clone OKT4), CD8 (clone SKI), CD45RO (clone UCHL1), CD62L (clone DREG-56), and human Fc Receptor Blocking Solution (TruStain FcX™, cat #422302) were purchased from BioLegend. Fixable Viability Dye eFluor 506 (Cat #65-0866) was purchased from Thermo Fisher Scientific.

To study T cell surface marker expression, cells were stained with Fixable Viability Dye first, followed by Fc blocking and surface marker staining, following a standard procedure as described previously (45). To study T cell intracellular cytokine production, CD8 T cells or primary TIIs were stimulated with PMA (Cat #80055-400, VWR; 50 ng/ml) and Ionomycin (Cat #80056-892, VWR; 500 ng/ml) in the presence of GolgiStop (Cat #554724, BD Biosciences; 4 μl per 6 ml culture) for 4 hours. At the end of the culture, cells were collected and intracellular cytokine (i.e., IFN-γ and TNF-α) staining was performed using a Fixation/Permeabilization Solution Kit (Cat #554714, BD Biosciences) and following the manufacturer's instructions. To study T cell intracellular cytotoxicity molecule production, CD8 T cells or primary TIIs were collected and then directly subjected to intracellular Granzyme B staining using a Fixation/Permeabilization Solution Kit (BD Biosciences). These cells were co-stained with surface markers to identify CD8 T cells (gated as TCRβ⁺CD8⁺ cells in vitro or CD45.2⁺TCRβ⁺CD8⁺ cells in vivo) or OT1 cells (gated as CD45.2⁺CD8⁺ cells in vivo). Stained cells were analyzed by using a MACSQuant Analyzer 10 flow cytometer (Miltenyi Biotec). A FlowJo software (Tree Star) was used to analyze the data.

### Enzyme-Linked Immunosorbent Assay (ELISA)

To study T cell cytokine production, MACS-purified mouse CD8 T cells were cultured in C10 medium under indicated experimental conditions for up to 4 days. At indicated time points, cell culture supernatants were collected for cytokine ELISA analysis following a standard protocol from the BD Biosciences. The coating and biotinylated antibodies for the detection of mouse IFN-γ (coating antibody, cat #554424; biotinylated detection antibody, cat #554426) and IL-2 (coating antibody, cat #551216; biotinylated detection antibody, cat #554410) were purchased from BD Biosciences. The streptavidin-HRP conjugate (Cat #18410051) was purchased from Invitrogen. Mouse IFN-γ (Cat #575309) and IL-2 (Cat #575409) standards were purchased from BioLegend. The 3,3′,5,5′-Tetramethylbenzidine (TMB, cat #51200048) substrate was purchased from KPL. The absorbance at 450 nm was measured using an Infinite M1000 microplate reader (Tecan).

To study T cell serotonin production, MACS-purified mouse CD8 T cells were cultured in C10 medium made of serotonin-depleted FBS and supplemented with L-Ascorbic acid, in the presence of plate-bound anti-mouse CD3ε (5 μg/ml) for up to 4 days. At indicated time points, cell culture supernatants were collected for serotonin ELISA analysis using a commercial kit following the manufacturer's instructions (SEU39-K01, Eagle Bioscience). The absorbance at 450 nm was measured using an Infinite M1000 microplate reader (Tecan).

### Western Blot (WB)

CD8 T cells purified from Maoa-WT and Maoa-KO mice were cultured in vitro in a 24-well plate at 0.5×10⁶ cells per well for 2 days, in the presence of plate-bound anti-mouse CD3ε (5 μg/ml), with or without asenapine treatment (10 μM). Cells were then rested on ice for 2 hours and restimulated with plate-bound anti-mouse CD3ε (5 μg/ml) for 20 minutes. Total protein was extracted using a lysis buffer containing 20 mM HEPES (pH 7.6), 150 mM NaCl, 1 mM EDTA, 1% TritonX-100, and protease/phosphatase inhibitor cocktail (Cat #5872S, Cell Signaling). Nuclear protein was extracted using a Nuclear Protein Extraction Kit (Cat #P178833, Thermo Fisher Scientific). Protein concentration was measured a BCA Assay Kit (Cat #23228 and Cat #1859078, Thermo Fisher Scientific). Equal amounts of protein were resolved on a 10% SDS-PAGE gel and then transferred to a PVDF membrane by electrophoresis. MAO-A antibody was purchased from Abcam (Cat #ab126751, Clone EPR7101). The following antibodies were purchased from the Cell Signaling and used to blot for the proteins of interest: anti-mouse NF-κB p65 (Cat #8242P, Clone D14E12), anti-mouse c-Jun (Cat #9165S, Clone 60A8), anti-mouse NFAT (Cat #4389S), anti-mouse ERK1/2 (Cat #9107S, Clone 3A7), anti-mouse p-ERK1/2 (Cat #43705, Clone D13.14.4E), secondary anti-mouse (Cat #7076P2), and secondary anti-rabbit (Cat #7074P2). β-actin (Cat #sc-69879, Clone AC-15, Santa Cruz Biotechnology) was used as an internal control for total protein extracts, while Lamin A/C (Cat #39287, Clone 3A6-4C11, Active Motif) was used as an internal control for nuclear protein extracts. Signals were visualized with autoradiography using an ECL system (Cat #RPN2232, Thermo Fisher Scientific). The data were analyzed using an Image Lab software (Bio-Rad).

### High Performance Liquid Chromatography (HPLC)

HPLC was used to measure intratumoral and serum serotonin levels as previously described (47, 48). Briefly, tumors and sera were collected from experimental mice at indicated time points, and were snap-frozen using liquid nitrogen. Frozen samples were thawed and homogenized using methanol and acetonitrile by vortexing. Homogenized samples were centrifuged, and supernatants were collected to new tubes and evaporated under a stream of argon. Dried sample pellets were then reconstituted in HPLC running buffer and were ready for analysis. Serotonin concentration was quantified using a C 18 column by reverse phase HPLC (System Gold 166P detector, Beckman Coulter)

Messenger RNA Quantitative RT-PCR (mRNA QPCR)

Total RNA was isolated using TRIzol Reagent (Cat #15596018, Invitrogen, Thermo Fisher Scientific) according to the manufacturer' instructions. cDNA was prepared using a SuperScript III First-Strand Synthesis Supermix Kit (Cat #18080400, Invitrogen, Thermo Fisher Scientific). Gene expression was measured using a KAPA SYBR FAST qPCR Kit (Cat #KM4117, Kapa Biosystems) and a 7500 Real-time PCR System (Applied Biosystems) according to the manufacturers' instructions. Ube2d2 was used as an internal control for mouse immune cells, and ACTIN was used as an internal control for human immune cells. The relative expression of the mRNA of interest was calculated using the 2^{ΔΔCT} method.

Single Cell RNA Sequencing (scRNASeq)

scRNASeq was used to analyze the gene expression profiling of TIIs. Day-14 B16-OVA tumors were harvested from Maoa-WT and Maoa-KO mice to prepare TII suspensions (10 tumors were combined for each group). TII suspensions were then sorted using a FACSAria II flow cytometer to purify immune cells (gated as DAPI⁻ CD45.2⁺ cells). Sorted TIIs were immediately delivered to the Technology Center for Genomics & Bioinformatics (TCGB) facility at UCLA for library construction and sequencing. Briefly, purified TIIs were quantified using a Cell Countess II automated cell counter (Invitrogen/Thermo Fisher Scientific). 10,000 TIIs from each experimental group were loaded on the Chromium platform (10× Genomics) and libraries were constructed using a Chromium Single Cell 3′ Library & Gel Bead Kit V2 (Cat #PN-120237, 10× Genomics) according to the manufacture's instructions. Libraries were sequenced on an Illumina Novaseq, using a Novaseq 6000 S2 Reagent Kit (100 cycles; 20012862, Illumina). Data analysis was performed using a Cellranger Software Suite (10× Genomics). BCL files were extracted from the sequencer and used as inputs for the cellranger pipeline to generate the digital expression matrix for each sample. Then cellranger aggr command was used to aggregate the two samples into one digital expression matrix. The matrix was analyzed using Seurat, an R package designed for single cell RNA sequencing. Specifically, cells were first filtered to have at least 300 UMIs (unique molecular identifiers), at least 100 genes and at most 50% mitochondrial gene expression; only 1 cell did not pass the filter. The filtered matrix was normalized using the Seurat function NormalizeData. Variable genes were found using the Seurat function FindVariableGenes. The matrix was scaled to regress out the sequencing depth for each cell. Variable genes that had been previously identified were used in principle component analysis (PCA) to reduce the dimensions of the data. Following this, 13 PCs were used in tSNE to further reduce the dimensions to two. The same 13 PCs were also used to group the cells into different clusters by the Seurat function FindClusters. Next, marker genes were found for each cluster and used to define the cell types. Subsequently, 2 clusters of tumor-infiltrating CD8 T cells (identified by co-expression of CD8A and CD3D marker genes) were extracted and compared between the Maoa-WT and Maoa-KO samples.

### Tumor Immune Dysfunction and Exclusion (TIDE) Computational Method

TIDE analysis was performed as previously described (24, 49) (http://tide.dfci.harvard.edu/query/). Briefly, this method was used to study the association between the tumor-infiltrating CD8 T cell (cytotoxic T lymphocyte, CTL) level and overall patient survival in relation to the intratumoral MAOA gene expression level. For each patient cohort, tumor samples were divided into MAOA-high (samples with MAOA expression one standard deviation above the average) and MAOA-low (remaining samples) groups, followed by analyzing the association between the CTL levels and survival outcomes in each group. The CTL level was estimated as the average expression level of CD8A, CD8B, GZMA, GZMB, and PRF1. Each survival plot presented tumors in two subgroups: “CTL-high” group had above-average CTL values among all samples, while ‘CTL-low’ group had below-average CTL values. A T cell dysfunction score (z score) was calculated for each patient cohort, correlating the MAOA expression level with the beneficial effect of CTL infiltration on patient survival. A positive z score indicates that the expression of MAOA is negatively correlated with the beneficial effect of tumor-infiltrating CTL on patient survival. The P value indicates the comparison between the MAOA-low and MAOA-high groups, and was calculated by two-sided Wald test in a Cox-PH regression.

### Statistics

A GraphPad Prism 7 software (GraphPad Software) was used for the graphic representation and statistical analysis of the data. Pairwise comparisons were made using a 2-tailed Student's t test. Multiple comparisons were performed using an ordinary one-way ANOVA followed by Tukey's multiple comparisons test, or an RM two-way ANOVA followed by Sidak multiple comparisons test. Data are presented as the mean±SEM, unless otherwise indicated. A P value of less than 0.05 was considered significant. ns, not significant, *P<0.05, **P<0.01, ***P<0.001. The P values of violin plots were determined by Wilcoxon rank sum test. The P values of comparison between survival plots were calculated by testing the association between TIDE prediction scores and overall survival with the two-sided Wald test in a Cox-PH regression.

### REFERENCES

1. J. Couzin-Frankel, Breakthrough of the year 2013. Cancer immunotherapy. Science 342, 1432-1433 (2013).
2. S. A. Rosenberg, N. P. Restifo, Adoptive cell transfer as personalized immunotherapy for human cancer. Science 348, 62-68 (2015).
3. W. A. Lim, C. H. June, The Principles of Engineering Immune Cells to Treat Cancer. Cell 168, 724-740 (2017).
4. S. H. Baumeister, G. J. Freeman, G. Dranoff, A. H. Sharpe, Coinhibitory Pathways in Immunotherapy for Cancer. Annu Rev Immunol 34, 539-573 (2016).
5. D. B. Page, M. A. Postow, M. K. Callahan, J. P. Allison, J. D. Wolchok, Immune modulation in cancer with antibodies. Annu Rev Med 65, 185-202 (2014).
6. A. Ribas, Releasing the Brakes on Cancer Immunotherapy. N Engl J Med 373, 1490-1492 (2015).
7. M. Dougan, G. Dranoff, S. K. Dougan, Cancer Immunotherapy: Beyond Checkpoint Blockade. Annu Rev Canc Biol 3, 55-75 (2019).
8. J. C. Shih, K. Chen, M. J. Ridd, Monoamine oxidase: from genes to behavior. Annu Rev Neurosci 22, 197-217 (1999).
9. J. E. Pintar, X. O. Breakefield, Monoamine oxidase (MAO) activity as a determinant in human neurophysiology. Behav Genet 12, 53-68 (1982).
10. H. G. Brunner, M. Nelen, X. O. Breakefield, H. H. Ropers, B. A. van Oost, Abnormal behavior associated with a point mutation in the structural gene for monoamine oxidase A. Science 262, 578-580 (1993).
11. J. H. Meyer et al., Elevated monoamine oxidase a levels in the brain: an explanation for the monoamine imbalance of major depression. Arch Gen Psychiatry 63, 1209-1216 (2006).
12. J. Tiihonen et al., Genetic background of extreme violent behavior. Mol Psychiatry 20, 786-792 (2015).
13. A. Gibbons, American Association of Physical Anthropologists meeting. Tracking the evolutionary history of a “warrior” gene. Science 304, 818 (2004).
14. M. Bortolato, K. Chen, J. C. Shih, Monoamine oxidase inactivation: from pathophysiology to therapeutics. Adv Drug Deliv Rev 60, 1527-1533 (2008).
15. M. Bortolato et al., Social deficits and perseverative behaviors, but not overt aggression, in MAO-A hypomorphic mice. Neuropsychopharmacology 36, 2674-2688 (2011).
16. L. T. Nguyen, P. S. Ohashi, Clinical blockade of PD1 and LAG3—potential mechanisms of action. Nat Rev Immunol 15, 45-56 (2015).
17. E. J. Wherry, M. Kurachi, Molecular and cellular insights into T cell exhaustion. Nat Rev Immunol 15, 486-499 (2015).
18. Y. Chen, M. Leon-Ponte, S. C. Pingle, P. J. O'Connell, G. P. Ahern, T lymphocytes possess the machinery for 5-HT synthesis, storage, degradation and release. Acta Physiol (Oxf) 213, 860-867 (2015).
19. M. Leon-Ponte, G. P. Ahern, P. J. O'Connell, Serotonin provides an accessory signal to enhance T-cell activation by signaling through the 5-HT7 receptor. Blood 109, 3139-3146 (2007).
20. P. J. O'Connell et al., A novel form of immune signaling revealed by transmission of the inflammatory mediator serotonin between dendritic cells and T cells. Blood 107, 1010-1017 (2006).
21. M. Wimbiscus, O. Kostenko, D. Malone, MAO inhibitors: risks, benefits, and lore. Cleve Clin J Med 77, 859-882 (2010).
22. M. T. Bethune et al., Isolation and characterization of NY-ESO-1-specific T cell receptors restricted on various MHC molecules. Proc Natl Acad Sci USA 115, E10702-E10711 (2018).
23. Y. Zhu et al., Development of Hematopoietic Stem Cell-Engineered Invariant Natural Killer T Cell Therapy for Cancer. Cell Stem Cell 25, 542-557 e549 (2019).
24. P. Jiang et al., Signatures of T cell dysfunction and exclusion predict cancer immunotherapy response. Nat Med 24, 1550-1558 (2018).
25. D. T. Chen et al., Complementary strand microRNAs mediate acquisition of metastatic potential in colonic adenocarcinoma. J Gastrointest Surg 16, 905-912; discussion 912-903 (2012).
26. S. Rousseaux et al., Ectopic activation of germline and placental genes identifies aggressive metastasis-prone lung cancers. Sci Transl Med 5, 186ra166 (2013).
27. N. Cancer Genome Atlas Research et al., The Cancer Genome Atlas Pan-Cancer analysis project. Nat Genet 45, 1113-1120 (2013).
28. J. K. Stratford et al., A six-gene signature predicts survival of patients with localized pancreatic ductal adenocarcinoma. PLoS Med 7, e1000307 (2010).
29. T. N. Gide et al., Distinct Immune Cell Populations Define Response to Anti-PD-1 Monotherapy and Anti-PD-1/Anti-CTLA-4 Combined Therapy. Cancer Cell 35, 238-255 e236 (2019).
30. S. Talbot, S. L. Foster, C. J. Woolf, Neuroimmunity: Physiology and Pathology. Annu Rev Immunol 34, 421-447 (2016).
31. M. Levite, Nerve-driven immunity. Neurotransmitters and neuropeptides in the immune system. Springer Book, (2012).
32. C. H. Schein, Repurposing approved drugs on the pathway to novel therapies. Med Res Rev, (2019).
33. G. Shoval et al., Adherence to antidepressant medications is associated with reduced premature mortality in patients with cancer: A nationwide cohort study. Depress Anxiety 36, 921-929 (2019).
34. P. M. Pilowsky, Serotonin—The Mediator That Spans Evolution. Elsevier Book, (2019).
35. M. E. Hernandez et al., Evaluation of the effect of selective serotonin-reuptake inhibitors on lymphocyte subsets in patients with a major depressive disorder. Eur Neuropsychopharmacol 20, 88-95 (2010).
36. G. J. McGinnis, J. Raber, CNS side effects of immune checkpoint inhibitors: preclinical models, genetics and multimodality therapy. Immunotherapy 9, 929-941 (2017).
37. A. H. Miller, C. L. Raison, The role of inflammation in depression: from evolutionary imperative to modern treatment target. Nat Rev Immunol 16, 22-34 (2016).
38. D. M. Pardoll, The blockade of immune checkpoints in cancer immunotherapy. Nat Rev Cancer 12, 252-264 (2012).
39. Y. C. Lin et al., MAOA—a novel decision maker of apoptosis and autophagy in hormone refractory neuroendocrine prostate cancer cells. Sci Rep 7, 46338 (2017).
40. S. Gaur, M. E. Gross, C. P. Liao, B. Qian, J. C. Shih, Effect of Monoamine oxidase A (MAOA) inhibitors on androgen-sensitive and castration-resistant prostate cancer cells. The Prostate 79, 667-677 (2019).
41. Y. Liu et al., In situ modulation of dendritic cells by injectable thermosensitive hydrogels for cancer vaccines in mice. Biomacromolecules 15, 3836-3845 (2014).
42. B. Homet Moreno et al., Response to Programmed Cell Death-1 Blockade in a Murine Melanoma Syngeneic Model Requires Costimulation, CD4, and CD8 T Cells. Cancer Immunol Res 4, 845-857 (2016).
43. S. Di Biase et al., Creatine uptake regulates CD8 T cell antitumor immunity. J Exp Med 216, 2869-2882 (2019).
44. D. J. Smith et al., Genetic engineering of hematopoietic stem cells to generate invariant natural killer T cells. Proc Natl Acad Sci USA 112, 1523-1528 (2015).
45. B. Li et al., miR-146a modulates autoreactive Th17 cell differentiation and regulates organ-specific autoimmunity. J Clin Invest 127, 3702-3716 (2017).
46. D. J. Smith et al., Propagating Humanized BLT Mice for the Study of Human Immunology and Immunotherapy. Stem Cells Dev 25, 1863-1873 (2016).
47. J. Thomas, R. Khanam, D. Vohora, A validated HPLC-UV method and optimization of sample preparation technique for norepinephrine and serotonin in mouse brain. Pharm Biol 53, 1539-1544 (2015).
48. T. M. Alshammari, A. A. Al-Hassan, T. B. Hadda, M. Aljofan, Comparison of different serum sample extraction methods and their suitability for mass spectrometry analysis. Saudi Pharm J 23, 689-697 (2015).
49. M. B. Dong et al., Systematic Immunotherapy Target Discovery Using Genome-Scale In Vivo CRISPR Screens in CD8 T Cells. Cell 178, 1189-1204 e1123 (2019).

All publications mentioned herein (e.g. Wang et al., Targeting monoamine oxidase A for T cell-based cancer immunotherapy; Sci Immunol. 2021 May 14; 6(59):eabh2383. doi: 10.1126/sciimmunol.abh2383. PMID: 33990379 and Wang et al., Targeting monoamine oxidase A-regulated tumor-associated macrophage polarization for cancer immunotherapy; Nat Commun. 2021 Jun. 10; 12(1):3530. doi: 10.1038/s41467-021-23164-2; and the references numerically listed above) are incorporated herein by reference to disclose and describe aspects, methods and/or materials in connection with the cited publications.

1. A composition of matter comprising:
a chemotherapeutic agent; and
a monoamine oxidase A inhibitor.

2. The composition of claim 1, wherein a monoamine oxidase A inhibitor comprises at least one of:
phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone.

3. The composition of claim 1, wherein;
the composition comprises a pharmaceutically acceptable carrier;
the composition comprises a lipid; and/or
the composition comprises the monoamine oxidase A inhibitor disposed within a nanoparticle.

4. The composition of claim 1, wherein the monoamine oxidase A inhibitor is present in the composition in amounts such that amounts of monoamine oxidase A inhibitor available for CD8 T cells in an individual administered the composition are sufficient to modulate the phenotype of the CD8 T cells.

5. The composition of claim 4, wherein modulation of the phenotype of the CD8 T cells comprises at least one of: enhanced tumor immunoreactivity; enhanced secretion of serotonin; increased expression of IFN- [symbol] ; increased expression of Granzyme B; or decreased expression of PD-1.

6. The composition of claim 1, wherein the chemotherapeutic agent comprises:
an antibody;
carboplatin;
paclitaxel; or
at least one immune checkpoint inhibitor selected to affect CTLA-4 or a PD-1/PD-L1 blockade.

6. (canceled)

7. The composition of claim 6, wherein the antibody comprises at least one of:
pembrolizumab;
nivolumab;
atezolizumab;
avelumab;
bevacizumab; and
durvalumab.

8. A method of modulating a phenotype of a tumor-infiltrating CD8 T cell comprising introducing a monoamine oxidase A inhibitor in the environment in which the CD8 T cell is disposed; wherein amounts of the monoamine oxidase A inhibitor introduced into the environment are selected to be sufficient to modulate the phenotype of the tumor-infiltrating CD8 T cell.

9. The method of claim 8, wherein the tumor-infiltrating CD8 T cell is disposed in an individual diagnosed with cancer.

10. The method of claim 9, wherein the individual is undergoing a therapeutic regimen comprising the administration of a chemotherapeutic agent.

11. The method of claim 9, wherein the cancer is a lymphoma or a skin, breast, ovarian, prostate, colorectal or lung cancer.

12. The method of claim 8, wherein modulation of the phenotype of the tumor-infiltrating CD8 T cell comprises at least one of: enhanced tumor immunoreactivity; enhanced secretion of serotonin; increased expression of IFN- [symbol] ; increased expression of Granzyme B; or decreased expression of PD-1.

13. The method of claim 8, wherein the monoamine oxidase A inhibitor comprises at least one of:
phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone.

14. The method of claim 13, wherein the monoamine oxidase A inhibitor is disposed within a nanoparticle.

15. The method of claim 10, wherein the chemotherapeutic agent comprises:
an antibody;
carboplatin;
paclitaxel; or
at least one immune checkpoint inhibitor selected to affect CTLA-4 or a PD-1/PD-L1 blockade.

16. A method of treating a cancer in an individual comprising administering to the individual a monoamine oxidase A inhibitor; wherein amounts of the monoamine oxidase A inhibitor administered to the individual are selected to be sufficient to modulate the phenotype of tumor-infiltrating CD8 T cells in the individual.

17. The method of claim 16, wherein modulation of the phenotype of the tumor-infiltrating CD8 T cells comprises at least one of: enhanced tumor immunoreactivity; enhanced secretion of serotonin; increased expression of IFN- [symbol] ; increased expression of Granzyme B; or decreased expression of PD-1.

18. The method of claim 16, wherein the individual is undergoing a therapeutic regimen comprising the administration of at least one chemotherapeutic agent.

19. The method of claim 16, wherein the cancer is a lymphoma or a skin, breast, ovarian, prostate, colorectal or lung cancer.

20. The method of claim 8, wherein the monoamine oxidase A inhibitor is disposed within a composition comprising a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and the monoamine oxidase A inhibitor disposed within the liposome.
